(19) United States
(12) Patent Application Publication
(10) Pub. No.: US 2005/0209251 A1
(43) Pub. Date: Sep. 22, 2005

(54) Substituted pyrazolo-pyrimidine-4-ones

(76) Inventors: Karl-Heinz Linker, Leverkusen (DE); Roland Andree, Langenfeld (DE); Dorothee Heischen, Dusseldorf (DE); Hans-Georg Schwarz, Langenfeld (DE); Mark-Wilhelm Drewes, Langenfeld (DE); Peter Dahmen, Neuss (DE); Dieter Feucht, Monheim (DE); Rolf Pontzen, Leichlingen (DE); Peter Losel, Leverkusen (DE)

Correspondence Address: BAYER CROPSCIENCE LP;Patent Department, 100 BAYER ROAD, PITTSBURGH, PA, 15205-9741, US

(21) Appl. No.: 10/512,834

(22) PCT Filed: Apr. 22, 2003
(86) PCT No.: PCT/EP03/04137

§ 371 Date: May 19, 2005

(30) Foreign Application Priority Data
May 2, 2002 (DE) 10219435.1

(51) Int. Cl. A01N 43/90; C07D 487/02

(52) U.S. Cl. 514/262.1; 544/262; 504/241

(57) ABSTRACT

The invention relates to compounds of the formula (I) 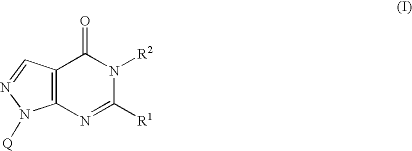 in which
Q, R¹ and R² are as defined in the description, to a process for the preparation and to their use as herbicides and/or nematicides.

The invention relates to novel substituted pyrazolopyrimidin-4-ones, to processes for their preparation and to their use as crop treatment agents, in particular as herbicides and as nematicides.

Certain substituted pyrazolopyrimidin-4-ones, such as, for example, the compound 1,5-dihydro-6-methyl-1-(2,4,6-trichlorophenyl)-4H-pyrazolo-[3,4-d]-pyrimidin-4-one, are already known (cf. WO 94/13677, U.S. Pat. No. 6,218,397). However, these compounds have not attained any importance as crop treatment agents.

This invention now provides novel substituted pyrazolopyrimidin-4-ones of the general formula (I) 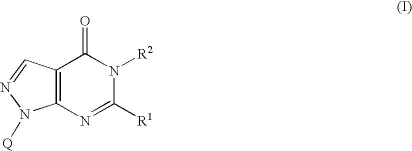
in which
Q represents aryl or heteroaryl, each of which is substituted by at least two identical or different substituents from the group consisting of nitro, cyano, halogen and in each case optionally halogen-substituted C₁-C₆-alkyl, C₁-C₆-alkoxy, C₁-C₆-alkylthio, C₁-C₆-alkylsulphinyl or C₁-C₆-alkylsulphonyl and each of which has up to 10 carbon atoms and, if appropriate, up to 5 nitrogen atoms and/or, if appropriate, one oxygen or sulphur atom,
R¹ represents hydrogen, represents in each case optionally cyano-, halogen- or C₁-C₄-alkoxy-substituted C₁-C₆-alkyl or C₁-C₆-alkoxycarbonyl, or represents in each case optionally halogen-substituted C₂-C₆-alkenyl or C₂-C₆-alkynyl, represents in each case optionally cyano-, halogen- or C₁-C₄-alkyl-substituted C₃-C₆-cycloalkyl or C₃-C₆-cycloalkyl-C₁-C₄-alkyl, represents in each case optionally nitro-, cyano-, halogen-, C₁-C₄-alkyl-, C₁-C₄-haloalkyl-, C₁-C₄-alkoxy- or C₁-C₄-haloalkoxy-substituted aryl or arylalkyl having in each case up to 10 carbon atoms in the aryl group and, if appropriate, up to 4 carbon atoms in the alkyl moiety, or represents optionally nitro-, cyano-, halogen-, C₁-C₄-alkyl-, C₁-C₄-haloalkyl-, C₁-C₄-alkoxy- or C₁-C₄-haloalkoxy-substituted heterocyclyl having up to 10 carbon atoms, up to 5 nitrogen atoms and/or one oxygen or sulphur atom, and
R² represents hydrogen, represents optionally cyano-, halogen-, C₁-C₄-alkoxy- or C₁-C₄-alkoxy-carbonyl-substituted C₁-C₆-alkyl or represents in each case optionally halogen-substituted C₂-C₆-alkenyl or C₂-C₆-alkynyl,
   except for the prior-art compound 1,5-dihydro-6-methyl-1-(2,4,6-trichlorophenyl)-4H-pyrazolo-[3,4-d]-pyrimidin-4-one (cf. WO 94/13677), which is excluded by disclaimer.

The present invention also provides the pyrazolopyrimidines of the general formula (Ia) 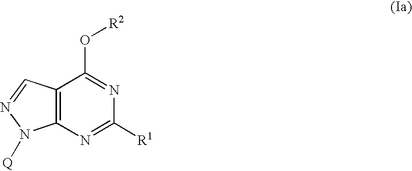
in which
Q, R¹ and R² are as defined above,
   which are isomeric to the substituted pyrazolopyrimidin-4-ones of the general formula (1).

In the definitions, the hydrocarbon chains, such as alkyl or alkenyl, are in each case straight-chain or branched—even in combination with heteroatoms, such as in alkoxy.

Optionally substituted radicals can be mono- or polysubstituted; and in the case of polysubstitution, the substituents can be identical or different.

Preferred substituents or ranges of the radicals present in the formulae listed above and below are as defined below:
Q preferably represents aryl having 6 or 10 carbon atoms or heteroaryl having up to 5 carbon atoms, up to 3 nitrogen atoms and/or, if appropriate, one oxygen or sulphur atom, each of which radicals is substituted by at least two identical or different substituents from the group consisting of nitro, cyano, fluorine, chlorine, bromine and C₁-C₄-alkyl, C₁-C₄-alkoxy, C₁-C₄-alkylthio, C₁-C₄-alkylsulphinyl and C₁-C₄-alkylsulphonyl, each of which is optionally substituted by 1 to 3 fluorine and/or chlorine atoms.
R¹ preferably represents hydrogen, represents in each case optionally cyano-, fluorine-, chlorine-, methoxy-, ethoxy-, n- or i-propoxy-substituted C₁-C₅-alkyl or C₁-C₅-alkoxycarbonyl, represents in each case optionally fluorine-, chlorine- and/or bromine-substituted C₂-C₅-alkenyl or C₂-C₅-alkynyl, represents in each case optionally cyano-, fluorine-, chlorine-, methyl- or ethyl-substituted C₃-C₆-cycloalkyl or C₃-C₆-cycloalkyl-C₁-C₃-alkyl, represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted aryl or arylalkyl having in each case 6 or 10 carbon atoms in the aryl group and, if appropriate, up to 3 carbon atoms in the alkyl moiety, or represents optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted heterocyclyl having up to 10 carbon atoms, up to 4 nitrogen atoms and/or one oxygen or sulphur atom.
R² preferably represents hydrogen, represents optionally cyano-, fluorine-, chlorine-, bromine-, methoxy-, ethoxy-, n- or i-propoxy-, methoxycarbonyl-, ethoxycarbonyl-, n- or i-propoxycarbonyl-substituted C₁-C₅-alkyl or represents in each case optionally fluorine-, chlorine- and/or bromine-substituted C₂-C₅-alkenyl or C₂-C₅-alkynyl.
Q particularly preferably represents phenyl, pyridinyl, pyrimidinyl, pyrazolyl, furyl or thienyl, each of which is substituted by at least two identical or different substituents from the group consisting of nitro, cyano, fluorine, chlorine, bromine, methyl, ethyl, n- or i-propyl, n-, i-, s- or t-butyl, fluoromethyl, chloromethyl, bromomethyl, difluoromethyl, dichloromethyl, trifluoromethyl, trichloromethyl, chlorodifluoromethyl, fluorodichloromethyl, fluoroethyl, chloroethyl, bromoethyl, difluoroethyl, dichloroethyl, chlorofluoroethyl, trifluoroethyl, trichloroethyl, chlorodifluoroethyl, fluorodichloroethyl, tetrafluoroethyl, pentafluoroethyl, methoxy, ethoxy, n- or i-propoxy, difluoromethoxy, trifluoromethoxy, chlorodifluoromethoxy, fluorodichloromethoxy, fluoroethoxy, chloroethoxy, difluoroethoxy, dichloroethoxy, chlorofluoroethoxy, trifluoroethoxy, methylthio, ethylthio, n- or i-propylthio, difluoromethylthio, trifluoromethylthio, chlorodifluoromethylthio, fluorodichloromethylthio, methylsulphinyl, ethylsulphinyl, trifluoromethylsulphinyl, methylsulphonyl, ethylsulphonyl or trifluoromethylsulphonyl.
R¹ particularly preferably represents hydrogen, represents in each case optionally cyano-, fluorine-, chlorine-, methoxy-, ethoxy-, n- or i-propoxy-substituted methyl, ethyl, n- or i-propyl, n-, i-, s- or t-butyl, methoxycarbonyl, ethoxycarbonyl, n- or i-propoxycarbonyl, n-, i-, s- or t-butoxycarbonyl, represents in each case optionally fluorine-, chlorine- and/or bromine-substituted ethenyl, propenyl, butenyl, pentenyl, ethynyl, propynyl, butynyl or pentynyl, represents in each case optionally cyano-, fluorine-, chlorine-, methyl- or ethyl-substituted cyclopropyl, cyclobutyl, cyclopentyl, cyclohexyl, cyclopropylmethyl, cyclobutylmethyl, cyclopentylmethyl or cyclohexylmethyl, represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted phenyl, benzyl or phenylethyl, or represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted pyridinyl, pyrimidinyl, furyl, tetrahydrofuryl or thienyl.
R² particularly preferably represents hydrogen, represents in each case optionally cyano-, fluorine-, chlorine-, bromine-, methoxy-, ethoxy-, n- or i-propoxy-, methoxycarbonyl-, ethoxycarbonyl-, n- or i-propoxycarbonyl-substituted methyl, ethyl, n- or i-propyl, n-, i-, s- or t-butyl, or represents in each case optionally fluorine-, chlorine- and/or bromine-substituted ethenyl, propenyl, butenyl, pentenyl, ethynyl, propynyl, butynyl or pentynyl.
Q very particularly preferably represents phenyl, pyridinyl or pyrazolyl, each of which is substituted by at least two identical or different substituents from the group consisting of nitro, cyano, fluorine, chlorine, bromine, methyl, ethyl, difluoromethyl, dichloromethyl, trifluoromethyl, trichloromethyl, chlorodifluoromethyl, fluorodichloromethyl, methoxy, ethoxy, difluoromethoxy, trifluoromethoxy, chlorodifluoromethoxy, fluorodichloromethoxy, fluoroethoxy, chloroethoxy, difluoroethoxy, dichloroethoxy, chlorofluoroethoxy, trifluoroethoxy, methylthio, ethylthio, difluoromethylthio, trifluoromethylthio, chlorodifluoromethylthio, fluorodichloromethylthio, methylsulphinyl, ethylsulphinyl, trifluoromethylsulphinyl, methylsulphonyl, ethylsulphonyl and trifluoromethylsulphonyl.
R¹ very particularly preferably represents hydrogen, represents methyl, ethyl, n- or i-propyl, n-, i- or s-butyl, difluoromethyl, dichloromethyl, trifluoromethyl, trichloromethyl, chlorodifluoromethyl, fluorodichloromethyl, fluoroethyl, chloroethyl, difluoroethyl, dichloroethyl, chlorofluoroethyl, trifluoroethyl, tetrafluoroethyl, pentafluoroethyl, methoxymethyl, ethoxymethyl, methoxyethyl, ethoxyethyl, methoxycarbonyl, ethoxycarbonyl, n- or i-propoxycarbonyl, represents ethenyl, propenyl, butenyl, pentenyl, fluoropropenyl, chloropropenyl, difluoropropenyl, dichloropropenyl, chlorofluoropropenyl, fluorobutenyl, chlorobutenyl, difluorobutenyl, dichlorobutenyl, chlorofluorobutenyl, ethynyl, propynyl, butynyl or pentynyl, represents in each case optionally fluorine-, chlorine- or methyl-substituted cyclopropyl, cyclobutyl, cyclopentyl or cyclohexyl, represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted phenyl, benzyl or phenylethyl, or represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted pyridinyl, pyrimidinyl, furyl, tetrahydrofuryl or thienyl.
R² very particularly preferably represents hydrogen, represents in each case optionally cyano-, fluorine-, chlorine-, methoxy-, ethoxy-, methoxycarbonyl-, ethoxycarbonyl-, n- or i-propoxycarbonyl-substituted methyl, ethyl, n- or i-propyl, or represents in each case optionally fluorine- and/or chlorine-substituted propenyl, butenyl, pentenyl, propynyl, butynyl or pentynyl.

Preference according to the invention is given to the compounds of the formula (I) which contain a combination of the meanings listed above as being preferred.

Particular preference according to the invention is given to the compounds of the formula (I) which contain a combination of the meanings listed above as being particularly preferred.

Very particular preference according to the invention is given to the compounds of the formula (I) which contain a combination of the meanings listed above as being very particularly preferred.

Preference is also given to those compounds of the formula (I) in which Q is substituted by two radicals. A very particularly preferred group are also those compounds of the formula (I) in which
Q represents phenyl or pyridinyl, each of which is substituted by at least two identical or different substituents from the group consisting of nitro, cyano, fluorine, chlorine, bromine, methyl, ethyl, difluoromethyl, dichloromethyl, trifluoromethyl, trichloromethyl, chlorodifluoromethyl, fluorodichloromethyl, methoxy, ethoxy, difluoromethoxy, trifluoromethoxy, chlorodifluoromethoxy, fluorodichloromethoxy, fluoroethoxy, chloroethoxy, difluoroethoxy, dichloroethoxy, chlorofluoroethoxy, trifluoroethoxy, methylthio, ethylthio, difluoromethylthio, trifluoromethylthio, chlorodifluoromethylthio, fluorodichloromethylthio, methylsulphinyl, ethylsulphinyl, trifluoromethylsulphinyl, methylsulphonyl, ethylsulphonyl and trifluoromethylsulphonyl.

A very particularly preferred group are those compounds of the formula (I) in which
Q represents phenyl which contains at least two identical or different substituents in the 2- and 4-positions and optionally one further substituent in the 6-position, the substituents being selected from the group consisting of nitro, cyano, fluorine, chlorine, bromine, methyl, ethyl, difluoromethyl, dichloromethyl, trifluoromethyl, trichloromethyl, chlorodifluoromethyl, fluorodichloromethyl, methoxy, ethoxy, difluoromethoxy, trifluoromethoxy, chlorodifluoromethoxy, fluorodichloromethoxy, fluoroethoxy, chloroethoxy, difluoroethoxy, dichloroethoxy, chlorofluoroethoxy, trifluoroethoxy, methylthio, ethylthio, difluoromethylthio, trifluoromethylthio, chlorodifluoromethylthio, fluorodichloromethylthio, methylsulphinyl, ethylsulphinyl, trifluoromethylsulphinyl, methylsulphonyl, ethylsulphonyl and trifluoromethylsulphonyl,
R¹ represents hydrogen, represents methyl, ethyl, n- or i-propyl, n-, i- or s-butyl, difluoromethyl, dichloromethyl, trifluoromethyl, trichloromethyl, chlorodifluoromethyl, fluorodichloromethyl, fluoroethyl, chloroethyl, difluoroethyl, dichloroethyl, chlorofluoroethyl, trifluoroethyl, tetrafluoroethyl, pentafluoroethyl, methoxymethyl, ethoxymethyl, methoxyethyl, ethoxyethyl, methoxycarbonyl, ethoxycarbonyl, n- or i-propoxycarbonyl, represents ethenyl, propenyl, butenyl, pentenyl, fluoropropenyl, chloropropenyl, difluoropropenyl, dichloropropenyl, chlorofluoropropenyl, fluorobutenyl, chlorobutenyl, difluorobutenyl, dichlorobutenyl, chlorofluorobutenyl, ethynyl, propynyl, butynyl or pentynyl, represents in each case optionally fluorine-, chlorine- or methyl-substituted cyclopropyl, cyclobutyl, cyclopentyl or cyclohexyl, represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted phenyl, benzyl or phenylethyl, or represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted pyridinyl, pyrimidinyl, furyl, tetrahydrofuryl or thienyl, and
R² represents hydrogen, represents in each case optionally cyano-, fluorine-, chlorine-, methoxy-, ethoxy-, methoxycarbonyl-, ethoxycarbonyl-, n- or i-propoxycarbonyl-substituted methyl, ethyl, n- or i-propyl, or represents in each case optionally fluorine- and/or chlorine-substituted propenyl, butenyl, pentenyl, propynyl, butynyl or pentynyl.

Here, very particular emphasis is given to those compounds in which Q represents 2,4-dichlorophenyl, 2,4,6-trichlorophenyl, 2-chloro-4-trifluoromethylphenyl or 2,6-dichloro-4-trifluoromethylphenyl.

A further very particularly preferred group are those compounds of the formula (I) in which
Q represents pyridin-2-yl which contains at least two identical or different substituents in the 3- and 5-positions and optionally one further substituent in the 6-position, the substituents being selected from the group consisting of nitro, cyano, fluorine, chlorine, bromine, methyl, ethyl, difluoromethyl, dichloromethyl, trifluoromethyl, trichloromethyl, chlorodifluoromethyl, fluorodichloromethyl, methoxy, ethoxy, difluoromethoxy, trifluoromethoxy, chlorodifluoromethoxy, fluorodichloromethoxy, fluoroethoxy, chloroethoxy, difluoroethoxy, dichloroethoxy, chlorofluoroethoxy, trifluoroethoxy, methylthio, ethylthio, difluoromethylthio, trifluoromethylthio, chlorodifluoromethylthio, fluorodichloromethylthio, methylsulphinyl, ethylsulphinyl, trifluoromethylsulphinyl, methylsulphonyl, ethylsulphonyl and trifluoromethylsulphonyl,
R¹ represents hydrogen, represents methyl, ethyl, n- or i-propyl, n-, i- or s-butyl, difluoromethyl, dichloromethyl, trifluoromethyl, trichloromethyl, chlorodifluoromethyl, fluorodichloromethyl, fluoroethyl, chloroethyl, difluoroethyl, dichloroethyl, chlorofluoroethyl, trifluoroethyl, tetrafluoroethyl, pentafluoroethyl, methoxymethyl, ethoxymethyl, methoxyethyl, ethoxyethyl, methoxycarbonyl, ethoxycarbonyl, n- or i-propoxycarbonyl, represents ethenyl, propenyl, butenyl, pentenyl, fluoropropenyl, chloropropenyl, difluoropropenyl, dichloropropenyl, chlorofluoropropenyl, fluorobutenyl, chlorobutenyl, difluorobutenyl, dichlorobutenyl, chlorofluorobutenyl, ethynyl, propynyl, butynyl or pentynyl, represents in each case optionally fluorine-, chlorine- or methyl-substituted cyclopropyl, cyclobutyl, cyclopentyl or cyclohexyl, represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted phenyl, benzyl or phenylethyl, or represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted pyridinyl, pyrimidinyl, furyl, tetrahydrofuryl or thienyl, and
R² represents hydrogen, represents in each case optionally cyano-, fluorine-, chlorine-, methoxy-, ethoxy-, methoxycarbonyl-, ethoxycarbonyl-, n- or i-propoxycarbonyl-substituted methyl, ethyl, n- or i-propyl, or represents in each case optionally fluorine- and/or chlorine-substituted propenyl, butenyl, pentenyl, propynyl, butynyl or pentynyl.

Here, very particular emphasis is given to those compounds in which Q represents 3,5-dichloropyridin-2-yl or 3-chloro-5-trifluoromethylpyridin-2-yl.

A further very particularly preferred group are those compounds of the formula (I) in which
Q represents pyrazol-3-yl which contains at least two identical or different substituents in the 1- and 5-positions and optionally one further substituent in the 4-position, the substituents being selected from the group consisting of nitro, cyano, fluorine, chlorine, bromine, methyl, ethyl, difluoromethyl, dichloromethyl, trifluoromethyl, trichloromethyl, chlorodifluoromethyl, fluorodichloromethyl, methoxy, ethoxy, difluoromethoxy, trifluoromethoxy, chlorodifluoromethoxy, fluorodichloromethoxy, fluoroethoxy, chloroethoxy, difluoroethoxy, dichloroethoxy, chlorofluoroethoxy, trifluoroethoxy, methylthio, ethylthio, difluoromethylthio, trifluoromethylthio, chlorodifluoromethylthio, fluorodichloromethylthio, methylsulphinyl, ethylsulphinyl, trifluoromethylsulphinyl, methylsulphonyl, ethylsulphonyl and trifluoromethylsulphonyl,
R¹ represents hydrogen, represents methyl, ethyl, n- or i-propyl, n-, i- or s-butyl, difluoromethyl, dichloromethyl, trifluoromethyl, trichloromethyl, chlorodifluoromethyl, fluorodichloromethyl, fluoroethyl, chloroethyl, difluoroethyl, dichloroethyl, chlorofluoroethyl, trifluoroethyl, tetrafluoroethyl, pentafluoroethyl, methoxymethyl, ethoxymethyl, methoxyethyl, ethoxyethyl, methoxycarbonyl, ethoxycarbonyl, n- or i-propoxycarbonyl, represents ethenyl, propenyl, butenyl, pentenyl, fluoropropenyl, chloropropenyl, difluoropropenyl, dichloropropenyl, chlorofluoropropenyl, fluorobutenyl, chlorobutenyl, difluorobutenyl, dichlorobutenyl, chlorofluorobutenyl, ethynyl, propynyl, butynyl or pentynyl, represents in each case optionally fluorine-, chlorine- or methyl-substituted cyclopropyl, cyclobutyl, cyclopentyl or cyclohexyl, represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t--butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted phenyl, benzyl or phenylethyl, or represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted pyridinyl, pyrimidinyl, furyl, tetrahydrofuryl or thienyl, and
R² represents hydrogen, represents in each case optionally cyano-, fluorine-, chlorine-, methoxy-, ethoxy-, methoxycarbonyl-, ethoxycarbonyl-, n- or i-propoxycarbonyl-substituted methyl, ethyl, n- or i-propyl, or represents in each case optionally fluorine- and/or chlorine-substituted propenyl, butenyl, pentenyl, propynyl, butynyl or pentynyl.

Here, very particular emphasis is given to those compounds in which Q represents 5-difluoromethoxy-1-methylpyrazol-3-yl or 5-difluoromethoxy-1,4-dimethylpyrazol-3-yl.

The abovementioned general or preferred radical definitions apply both to the end products of the formula (I) and, correspondingly, to the starting materials or intermediates required in each case for the preparation. These radical definitions can be combined with one another as desired, i.e. including combinations between the given preferred ranges.

The novel substituted pyrazolopyrimidin-4-ones of the general formula (I) have strong and selective herbicidal and nematicidal activity.

The novel substituted pyrazolopyrimidin-4-ones of the general formula (I) are obtained when
(a) 5-amino-1-arylpyrazol-4-carboxamides of the general formula (II) 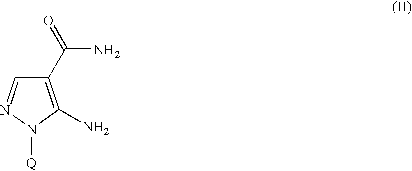
   in which
Q is as defined above,
are reacted with carboxylic orthoesters of the general formula (III)
   R¹—(OR′)₃ (III)
   in which
R¹ is as defined above and
R′ represents alkyl,
if appropriate in the presence of one or more reaction auxiliaries and if appropriate in the presence of one or more diluents,
   or when
(b) 5-amino-1-arylpyrazol-4-carbonitriles of the general formula (IV) 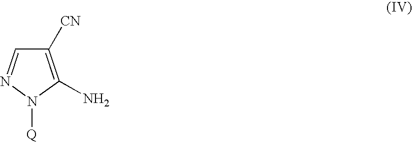
   in which
Q is as defined above
are reacted with carboxylic anhydrides of the general formula (V) 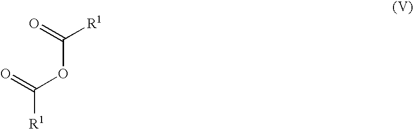
   in which
R¹ is as defined above,
if appropriate in the presence of one or more reaction auxiliaries and if appropriate in the presence of one or more diluents,
   or when
(c) 5-amino-1-arylpyrazol-4-carboxamides of the general formula (II) 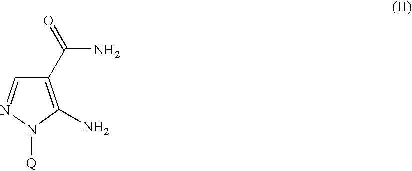
   in which
Q is as defined above,
   are reacted with carboxylic anhydrides of the general formula (V) 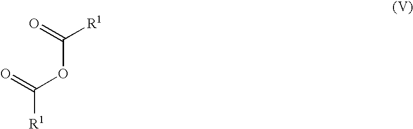
   in which
R¹ is as defined above,
if appropriate in the presence of one or more reaction auxiliaries and if appropriate in the presence of one or more diluents,
   or when
(d) 5-acylamino-1-arylpyrazole-4-carboxamides of the general formula (VI) 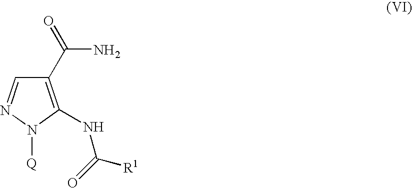
   in which
Q and R¹ are as defined above,
are reacted, if appropriate in the presence of one or more condensing agents and if appropriate in the presence of one or more diluents,
   or when
(e) substituted pyrazolopyrimidin-4-ones of the general formula (Ib) 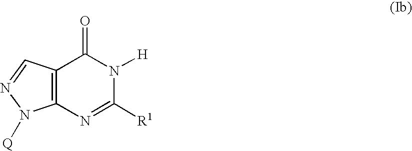
   in which
Q and R¹ are as defined above,
are reacted with alkylating, alkenylating or alkynylating agents of the general formula (VII)
   X—R² (VII)
   or of the general formula (VIII)
   R²—O—SO₂—O—R² (VIII)
   where in each case
R² is as defined above and
x represents halogen,
if appropriate in the presence of one or more reaction auxiliaries and if appropriate in the presence of one or more diluents.

Using, for example, 5-amino-1-(3,5-dichloropyridin-2-yl)-pyrazole-4-carboxamide and trimethyl orthoformate as starting materials, the course of the reaction in the process (a) according to the invention can be illustrated by the formula scheme below:

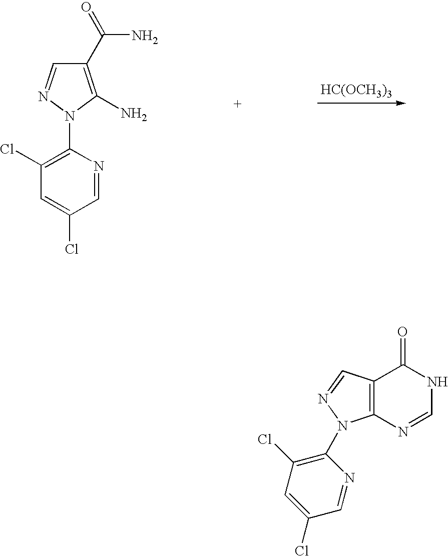

Using, for example, 5-amino-1-(3-chloro-5-trifluoromethylpyridin-2-yl)-pyrazole-4-carbonitrile and acetic anhydride as starting materials, the course of the reaction in the process (b) according to the invention can be illustrated by the formula scheme below:

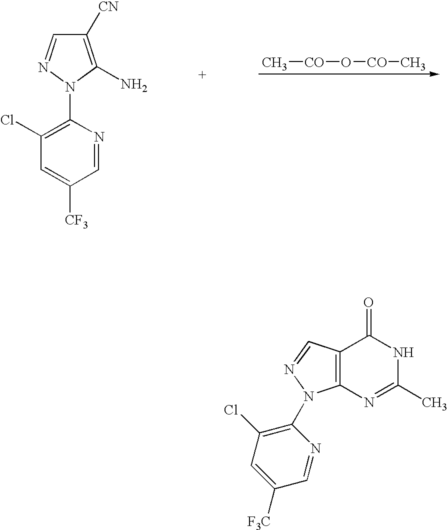

Using, for example, 5-amino-1-(2,4-dichlorophenyl)pyrazole-4-carboxamide and propionic anhydride as starting materials, the course of the reaction in the process (c) according to the invention can be illustrated by the formula scheme below:

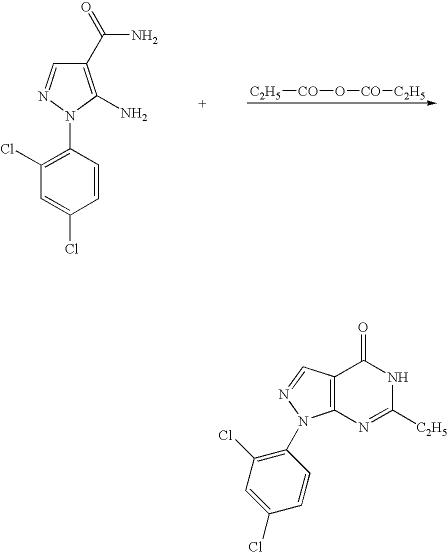

Using, for example, 1-(2-chloro-4-trifluoromethylphenyl)-5-trifluoro-acetylaminopyrazole-4-carboxamide as starting material, the course of the reaction in the process (d) according to the invention can be illustrated by the formula scheme below:

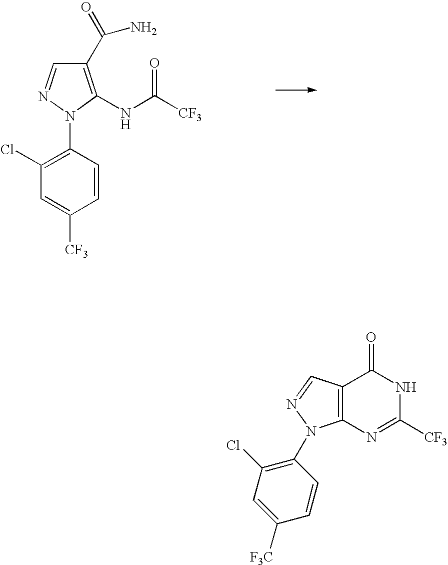

Using, for example, 1-(2,6-dichloro-4-trifluoromethylphenyl)-6-methyl-1,5-dihydropyrazolo[3,4-d]pyrimidin-4-one and methyl bromide as starting materials, the course of the reaction in the process (e) according to the invention can be illustrated by the formula scheme below:

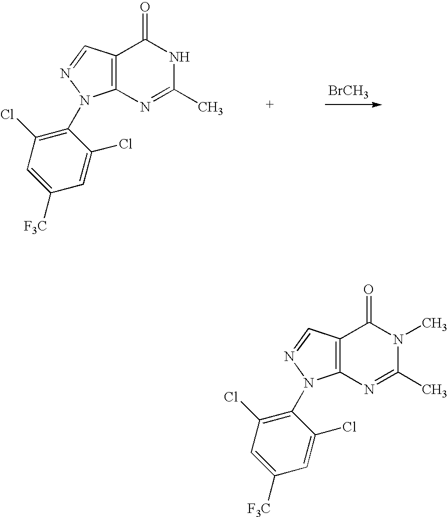

The formula (II) provides a general definition of the 5-amino-1-arylpyrazole-4-carboxamides to be used as starting materials in the processes (a) and (c) according to the invention for preparing compounds of the formula (I). In the formula (II), Q preferably has those meanings which have already been mentioned above, in connection with the description of the compounds of the formula (I) according to the invention, as being preferred, particularly preferred or very particularly preferred for Q.

Except for the compound 5-amino-1-(2,4,6-trichlorophenyl)-pyrazole-4-carboxamide (cf. WO-94/13677), the starting materials of the general formula (II) have, hitherto not been disclosed in the literature; except for the compound 5-amino-1-(2,4,6-trichlorophenyl)pyrazole-4-carboxamide, they form, as novel substances, part of the subject-matter of the present application.

The 5-amino-1-arylpyrazole-4-carboxamides of the general formula (II) are obtained when 5-amino-1-arylpyrazole-4-carbonitriles of the general formula (IV) 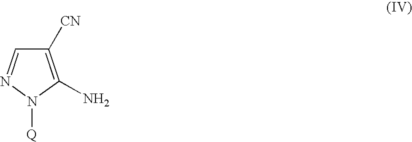
in which
Q is as defined above,
are hydrolysed in a customary manner, for example by reaction with sulphuric acid, at temperatures between 0° C. and 100° C. (cf. the Preparation Examples).

The formula (III) provides a general definition of the carboxylic ortho esters further to be used as starting materials in the process (a) according to the invention for preparing compounds of the formula (I). In the formula (III), R¹ preferably has those meanings which have already been mentioned above, in connection with the description of the compounds of the formula (I) according to the invention, as being preferred, particularly preferred or very particularly preferred for R¹; R′ preferably represents alkyl having 1 to 4 carbon atoms, in particular methyl or ethyl.

The starting materials of the general formula (III) are known organic chemicals for synthesis.

The formula (IV) provides a general definition of the 5-amino-1-arylpyrazole-4-carbonitriles to be used as starting materials in the process (b) according to the invention for preparing compounds of the formula (I). In the formula (IV), Q preferably has those meanings which have already been mentioned above, in connection with the description of the compounds of the formula (I) according to the invention, as being preferred, particularly preferred or very particularly preferred for Q.

The starting materials of the general formula (IV) are known and/or can be prepared by processes known per se (cf. DE 34 08 727, DE 34 20 985, DE 35 20 327, DE 35 20 331, DE 35 40 839, DE 36 25 686, DE 195 30 606, DE 196 23 892, DE 196 31 865, EP 542 388, GB 21 23 420, U.S. Pat. No. 5,167,691, U.S. Pat. No. 5,198,014, U.S. Pat. No. 5,250,504, WO 83/00331, WO 94/08999).

The formula (V) provides a general definition of the carboxylic anhydrides further to be used as starting materials in the processes (b) and (c) according to the invention for preparing compounds of the formula (1). In the formula (V), R¹ preferably has those meanings which have already been mentioned above, in connection with the description of the compounds of the formula (1) according to the invention, as being preferred, particularly preferred or very particularly preferred for R¹.

The starting materials of the general formula (V) are known organic chemicals for synthesis.

The formula (VI) provides a general definition of the 5-acylamino-1-arylpyrazole-4-carboxamides to be used as starting materials in the process (d) according to the invention for preparing compounds of the formula (I). In the formula (VI), Q and R¹ preferably have those meanings which have already been mentioned above, in connection with the description of the compounds of the formula (I) according to the invention, as being preferred, particularly preferred or very particularly preferred for Q and R¹.

Except for the compound 1-(2,6-dichloro-4-trifluoromethylthiophenyl)-5-propionyl-aminopyrazole-4-carboxamide (cf. DE 34 20 985), the starting materials of the general formula (VI) have hitherto not been disclosed in the literature; except for the compound 1-(2,6-dichloro-4-trifluoromethylthiophenyl)-5-propionylaminopyrazole-4-carboxamide, they form, as novel substances, part of the subject-matter of the present application.

The novel 5-acylamino-1-arylpyrazole-4-carboxamides of the formula (VI) are obtained when 5-amino-1-arylpyrazole-4-carboxamides of the general formula (II) 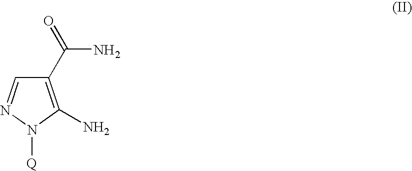
in which
Q is as defined above
are reacted with acylating agents of the general formula (IX)
   X¹—R¹ (IX)
   in which
R¹ is as defined above and
X¹ represents halogen, in particular fluorine, chlorine, or bromine,
if appropriate in the presence of a reaction auxiliary, such as, for example, sodium hydride, potassium carbonate or pyridine, and if appropriate in the presence of a diluent, such as, for example, acetonitrile, at temperatures between 0° C. and 100° C. (cf. the Preparation Examples).

The compounds of the formula (IX) used in the process are known chemicals for synthesis.

The formula (Ib) provides a general definition of the substituted pyrazolopyrimidin-4-ones to be used as starting materials in the process of (e) according to the invention for preparing compounds of the formula (I). In the formula (Ib), Q and R¹ preferably have those meanings which have already been mentioned above, in connection with the description of the compounds of the formula (I) according to the invention, as being preferred, particularly preferred or very particularly preferred for Q and R¹.

As novel substances, the starting materials of the general formula (Ib) form part of the subject-matter of the present application; they can be prepared by the processes (a) to (d) according to the invention.

The formulae (VII) and (VII) provide general definitions of the alkylating agents, alkenylating agents or alkynylating agents further to be used as starting materials in the process (e) according to the invention for preparing compounds of the formula (I). In the formulae (VII) and (VIII), R² preferably has those meanings which have already been mentioned above, in connection with the description of the compounds of the formula (I) according to the invention, as being preferred, particularly preferred or very particularly preferred for R²; X in formula (VII) preferably here represents fluorine, chlorine, bromine or iodine, in particular chlorine or bromine.

The starting materials of the formulae (VII) and (VIII) are known organic chemicals for synthesis.

The process (d) according to the invention is carried out using a condensing agent. Suitable condensing agents are especially basic compounds. These include in particular ammonia or amines, such as, for example, methylamine, ethylamine, n- or i-propylamine, n-, i-, s- or t-butylamine, dimethylamine, diethylamine, dipropylamine or dibutylamine, trimethylamine, triethylamine, tripropylamine or tributylamine, and also alkali metal or alkaline earth metal hydroxides, such as, for example, sodium hydroxide, potassium hydroxide, magnesium hydroxide or calcium hydroxide, or alkoxides, such as, for example, sodium methoxide, ethoxide, n- or i-propoxide, n-, i-, s- or t-butoxide or potassium methoxide, ethoxide, n- or i-propoxide, n-, i-, s- or t-butoxide.

The processes (a), (b), (c) and (e) according to the invention for preparing the compounds of the general formula (I) are preferably carried out using one or more reaction auxiliaries. Suitable reaction auxiliaries for the processes (a), (b), (c) and (e) according to the invention are, in general, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]octane (DABCO), 1,5-diazabicyclo[4.3.0]non-5-ene (DBN), or 1,8-diazabicyclo[5.4.0]undec-7-ene (DBU).

The processes (a) to (e) according to the invention for preparing the compounds of the general formula (I) are preferably carried out using one or more diluents. Suitable diluents for carrying out the processes according to the invention are especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile, propionitrile or butyronitrile; amides, such as N,N-dimethylformamide, N,N-dimethylacetamide, N-methylformanilide, N-methylpyrrolidone or hexamethylphosphoric triamide; esters, such as methyl acetate ethyl acetate; sulphoxides, such as dimethyl sulphoxide; alcohols, such as methanol, ethanol, n- or i-propanol, ethylene glycol monomethyl ether, ethylene glycol monoethyl ether, diethylene glycol monomethyl ether, diethylene glycol monoethyl ether, mixtures thereof with water or pure water.

When carrying out the processes according to the invention, the reaction temperatures can be varied within a relatively wide range. In general, the processes are carried out at temperatures between −30° C. and +150° C., preferably between 0° C. and 120° C.

The processes according to the invention are generally carried out under atmospheric pressure. However, it is also possible to carry out the processes according to the invention under elevated or reduced pressure—in general between 0.1 bar and 10 bar.

For carrying out the processes according to the invention, the starting materials are generally employed in approximately equimolar amounts. However, it is also possible to use a relatively large excess of one of the components. The reaction is generally carried out in a suitable diluent in the presence of a reaction auxiliary, and the reaction mixture is generally stirred at the required temperature for several hours. Work-up is carried out by customary methods (cf. the Preparation Examples).

The active compounds according to the invention can be used as defoliants, desiccants, haulm killers and, especially, as weed killers. Weeds in the broadest sense are understood to mean all plants which grow in locations where they are undesired. Whether the substances according to the invention act as total or selective herbicides depends essentially on the amount used.

The active compounds according to the invention can be used, for example, in connection with the following plants:

Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Dicotyledonous crops of the genera: Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea.

However, the use of the active compounds according to the invention is in no way restricted to these genera, but also extends in the same manner to other plants.

The active compounds according to the invention are suitable, depending on the concentration, for the total control of weeds, for example on industrial terrain and rail tracks, and on paths and areas with and without tree plantings. Similarly, the active compounds according to the invention can be employed for controlling weeds in perennial crops, for example forests, decorative tree plantings, orchards, vineyards, citrus groves, nut orchards, banana plantations, coffee plantations, tea plantations, rubber plantations, oil palm plantations, cocoa plantations, soft fruit plantings and hop fields, on lawns, turf and pastureland, and for the selective control of weeds in annual crops.

The compounds of the formula (I) according to the invention have strong herbicidal activity and a broad active spectrum when used on the soil and on above-ground parts of plants. To a certain extent they are also suitable for the selective control of monocotyledonous and dicotyledonous weeds in monocotyledonous and dicotyledonous crops, both by the pre-emergence and by the post-emergence method.

At certain concentrations or application rates, the active compounds according to the invention can also be employed for controlling animal pests and fungal or bacterial plant diseases. If appropriate, they can also be used as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and inclusive of the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

The treatment according to the invention of the plants and plant parts with the active compounds is carried out directly or by allowing the compounds to act on their surroundings, environment or storage space by the customary treatment methods, for example by immersion, spraying, evaporation, fogging, scattering, painting on and, in the case of propagation material, in particular in the case of seeds, also by applying one or more coats.

The active compounds can be converted into the customary formulations such as solutions, emulsions, wettable powders, suspensions, powders, dusts, pastes, soluble powders, granules, suspension-emulsion concentrates, natural and synthetic materials impregnated with active compound, and microencapsulations in polymeric materials.

These formulations are produced in a known manner, for example by mixing the active compounds with extenders, that is, liquid solvents and/or solid carriers, optionally with the use of surfactants, that is, emulsifiers and/or dispersants, and/or foam formers.

If the extender used is water, it is also possible, for example, to use organic solvents as cosolvents. The following are essentially suitable as liquid solvents: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water.

Suitable solid carriers are: for example ammonium salts and ground natural minerals such as kaolins, clays, talc, chalk, quartz, attapulgite, montmorillonite or diatomaceous earth, and ground synthetic minerals such as highly-disperse silica, alumina and silicates; suitable solid carriers for granules are: for example crushed and fractionated natural rocks such as calcite, marble, pumice, sepiolite and dolomite, or else synthetic granules of inorganic and organic minerals, and granules of organic material such as sawdust, coconut shells, maize cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates; suitable dispersants are: for example lignin-sulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other additives can be mineral and vegetable oils.

It is possible to use colorants such as inorganic pigments, for example iron oxide, titanium oxide and Prussian Blue, and organic colorants such alizarin colorants, azo colorants and metal phthalocyanine colorants, and trace nutrients such as salts of iron, manganese, boron, copper, cobalt, molybdenum and zinc.

The formulations generally comprise between 0.1 and 95 percent by weight of active compound, preferably between 0.5 and 90%.

For controlling weeds, the active compounds according to the invention, as such or in their formulations, can also be used as mixtures with known herbicides and/or substances which improve the compatibility with crop plants (“safeners”), finished formulations or tank mixes being possible. Also possible are mixtures with weed-killers comprising one or more known herbicides and a safener.

Suitable components for the mixtures are known herbicides, for example acetochlor, acifluorfen (-sodium), aclonifen, alachlor, alloxydim (-sodium), ametryne, amicarbazone, amidochlor, amidosulfuron, anilofos, asulam, atrazine, azafenidin, azimsulfuron, beflubutamid, benazolin (-ethyl), benfuresate, bensulfuron (-methyl), bentazone, benzfendizone, benzobicyclon, benzofenap, benzoylprop (-ethyl), bialaphos, bifenox, bispyribac (-sodium), bromobutide, bromofenoxim, bromoxynil, butachlor, butafenacil (-allyl), butroxydim, butylate, cafenstrole, caloxydim, carbetamide, carfentrazone (-ethyl), chlomethoxyfen, chloramben, chloridazone, chlorimuron (-ethyl), chlomitrofen, chlorsulfuron, chlortoluron, cinidon (-ethyl), cinmethylin, cinosulfuron, clefoxydim, clethodim, clodinafop (-propargyl), clomazone, clomeprop, clopyralid, clopyrasulfuron (-methyl), cloransulam (-methyl), cumyluron, cyanazine, cybutryne, cycloate, cyclosulfamuron, cycloxydim, cyhalofop (-butyl), 2,4-D, 2,4-DB, desmedipham, diallate, dicamba, dichlorprop (—P), diclofop (-methyl), diclosulam, diethatyl (-ethyl), difenzoquat, diflufenican, diflufenzopyr, dimefuron, dimepiperate, dimethachlor, dimethametryn, dimethenamid, dimexyflam, dinitramine, diphenamid, diquat, dithiopyr, diuron, dymron, epropodan, EPTC, esprocarb, ethalfluralin, ethametsulfuron (-methyl), ethofumesate, ethoxyfen, ethoxysulfuron, etobenzanid, fenoxaprop (—P-ethyl), fentrazamide, flamprop (-isopropyl, -isopropyl-L, -methyl), flazasulfuron, florasulam, fluazifop (—P-butyl), fluazolate, flucarbazone (-sodium), flufenacet, flufenpyr, flumetsulam, flumiclorac (-pentyl), flumioxazin, flumipropyn, flumetsulam, fluometuron, fluorochloridone, fluoroglycofen (-ethyl), flupoxam, flupropacil, flurpyrsulfuron (-methyl, -sodium), flurenol (-butyl), fluridone, fluroxypyr (-butoxypropyl, -meptyl), flurprimidol, flurtamone, fluthiacet (-methyl), fluthiamide, fomesafen, foramsulfuron, glufosinate (-ammonium), glyphosate (-isopropylammonium), halosafen, haloxyfop (-ethoxyethyl, —P-methyl), hexazinone, imazamethabenz (-methyl), imazamethapyr, imazamox, imazapic, imazapyr, imazaquin, imazethapyr, imazosulfuron, iodosulfuron (-methyl, -sodium), ioxynil, isopropalin, isoproturon, isouron, isoxaben, isoxachlortole, isoxaflutole, isoxapyrifop, ketospiradox, lactofen, lenacil, linuron, MCPA, mecoprop, mefenacet, mesotrione, metamitron, metazachlor, methabenzthiazuron, metobenzuron, metobromuron, (alpha) metolachlor, metosulam, metoxuron, metribuzin, metsulfuron (-methyl), molinate, monolinuron, naproanilide, napropamide, neburon, nicosulfuron, norflurazon, orbencarb, oryzalin, oxadiargyl, oxadiazon, oxasulfuron, oxaziclomefone, oxyfluorfen, paraquat, pelargonic acid, pendimethalin, pendralin, penoxysulam, pentoxazone, pethoxamid, phenmedipham, picolinafen, piperophos, pretilachlor, primisulfuron (-methyl), profluazol, profoxydim, prometryn, propachlor, propanil, propaquizafop, propisochlor, propoxycarbazone (-sodium), propyzamide, prosulfocarb, prosulfuron, pyraflufen (-ethyl), pyrazogyl, pyrazolate, pyrazosulfuron (-ethyl), pyrazoxyfen, pyribenzoxim, pyributicarb, pyridate, pyridatol, pyriftalid, pyriminobac (-methyl), pyrithiobac (-sodium), quinchlorac, quinmerac, quinoclamine, quizalofop (—P-ethyl, —P-tefuryl), rimsulfuron, sethoxydim, simazine, simetryn, sulcotrione, sulfentrazone, sulfometuron (-methyl), sulfosate, sulfosulfuron, tebutam, tebuthiuron, tepraloxydim, terbuthylazine, terbutryn, thenylchlor, thiafluamid, thiazopyr, thidiazimin, thifensulfuron (-methyl), thiobencarb, tiocarbazil, tralkoxydim, triallate, triasulfuron, tribenuron (-methyl), triclopyr, tridiphane, trifluralin, trifloxysulfuron, triflusulfuron (-methyl), tritosulfuron.

Furthermore suitable for the mixtures are known safeners, for example AD-67, BAS-145138, benoxacor, cloquintocet (-mexyl), cyometrinil, 2,4-D, DKA-24, dichlormid, dymron, fenclorim, fenchlorazol (-ethyl), flurazole, fluxofenim, furilazole, isoxadifen (-ethyl), MCPA, mecoprop (—P), mefenpyr (-diethyl), MG-191, oxabetrinil, PPG-1292, R-29148.

A mixture with other known active compounds, such as fungicides, insecticides, acaricides, nematicides, bird repellents, plant nutrients and agents which improve soil structure, is also possible.

The active compounds can be used as such, in the form of their formulations or in the use forms prepared therefrom by further dilution, such as ready-to-use solutions, suspensions, emulsions, powders, pastes and granules. They are used in a customary manner, for example by watering, spraying, atomizing or broadcasting.

The active compounds according to the invention can be applied both before and after emergence of the plants. They can also be incorporated into the soil before sowing.

The amount of active compound used can vary within a relatively wide range. It depends essentially on the nature of the desired effect. In general, the amounts used are between 1 g and 10 kg of active compound per hectare of soil surface, preferably between 5 g and 5 kg per ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are understood as meaning plants with novel properties (“traits”) which are grown by conventional cultivation, by mutagenesis or by recombinant DNA techniques. These may be cultivars, biotypes or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention—including in combination with other agrochemical active compounds, better growth of the crop plants, increased tolerance of the crop plants to high or low temperatures, increased tolerance of the crop plants to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparts particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits which are also particularly emphasized are the increased resistance of plants to fungi, bacteria and viruses by systemic acquired resistance (SAR), systemin, phytoalexins, elicitors and resistance genes and the correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready®D (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which cultivars will be developed and/or marketed in the future.

The plants listed can be treated according to the invention in a particularly advantageous manner with the compounds of the formula I or the active compound mixtures according to the invention, where, in addition to the effective control of the weed plants, the abovementioned synergistic effects with the transgenic plants or plant cultivars occur. The preferred ranges stated above for the active compounds or mixtures also apply to the treatment of these plants. Particular emphasis is given to the treatment of plants with the compounds or mixtures specifically mentioned in the present text.

Active compounds according to the invention are also suitable for controlling animal pests, in particular insects, arachnids and nematodes encountered in agriculture, in forests, in the protection of stored products and materials and in the hygiene sector.

They can preferably be used as crop protection agents. They are effective against normally sensitive and resistant species and against all or individual development stages.

When used as insecticides, acaricides or nematicides, the active compounds according to the invention can furthermore be present in their commercial formulations and in the use forms, prepared from these formulations, in a mixture with synergists. Synergists are compounds which enhance the activity of the active compounds, without it being necessary for the synergist added to be active for its part.

The content of active compound of the use forms prepared from the commercial formulations may vary within wide ranges. The concentration of active compound of the use forms can be from 0.0000001 to 95% by weight of active compound and is preferably from 0.0001 to 1% by weight.

The application is carried out in a manner suitable for the use forms.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100 to 10 000-fold dilution, or they can be used as a chemical bath.

The active compounds are also suitable for controlling animal pests, in particular insects, arachnids and mites, encountered in closed rooms, such as, for example, flats, factory buildings, offices, vehicle cabins and the like. For controlling these pests, they can be used on their own or in combination with other active compounds and auxiliaries in household insecticide products. They are active against sensitive and resistant species and against all stages of development.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free or passive evaporation systems, moth papers, moth bags and moth gels, as granules or dusts, in baits for spreading or in bait stations.

The preparation and the use of the active compounds according to the invention is illustrated by the examples below.

### PREPARATION EXAMPLES

### Example 1

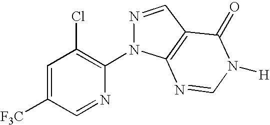 (Process (a))

A mixture of 0.70 g (2.29 mmol) of 5-amino-1-(3-chloro-5-trifluoromethylpyridin-2-yl)pyrazole-4-carboxamide, 0.27 g (2.52 mmol) of trimethyl orthoformate, 0.10 g of p-toluenesulphonic acid and 80 ml of toluene is stirred under reflux for 12 hours. A further 0.14 g of trimethyl orthoformate is then added to this mixture, and the mixture is stirred under reflux for another 12 hours. After cooling, the mixture is filtered and the filtrate is concentrated under reduced pressure. The residue is triturated with isopropanol and the resulting crystalline product is isolated by filtration with suction.

This gives 0.32 g (44% of theory) of 1-(3-chloro-5-trifluoromethylpyridin-2-yl)-1,5-dihydropyrazolo[3,4-d]pyrimidin-4-one of melting point 331° C.

logP (pH 2): 1.54

### Example 2

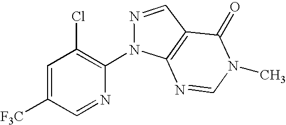 (Process (e))

A mixture of 0.21 g (0.665 mmol) of 1-(3-chloro-5-trifluoromethylpyridin-2-yl)-1,5-dihydropyrazolo[3,4-d]pyrimidin-4-one, 0.11 g (0.80 mmol) of potassium carbonate, 0.10 g (0.73 mmol) of methyl iodide and 40 ml of acetonitrile is stirred at 20° C.-25° C. for 12 hours and then concentrated under reduced pressure. The residue is then stirred with water and acidified with conc. hydrochloric acid, and the resulting crystalline product is isolated by filtration with suction.

This gives 0.12 g (55% of theory) of 1-(3-chloro-5-trifluoromethylpyridin-2-yl)-5-methyl-1,5-dihydropyrazolo[3,4-d]pyrimidin-4-one of melting point 253° C.

logP (pH 2): 1.76

### Example 3

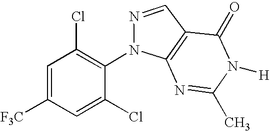 (Process (c))

A mixture of 1.36 g (4.0 mmol) of 5-amino-1-(2,6-dichloro-4-trifluoromethylphenyl)pyrazole-4-carboxamide, 0.61 g (6 mmol) of acetic anhydride and 50 ml of xylene is stirred at reflux temperature for 8 hours and then concentrated under reduced pressure. The oily residue is triturated with n-propanol, and the resulting crystalline product is isolated by filtration with suction.

This gives 0.48 g (30% of theory) of 1-(2,6-dichloro-4-trifluoromethylphenyl)-6-methyl-1,5-dihydropyrazolo[3,4-d]pyrimidin-4-one of melting point 300° C. logP (pH 2): 2.25

### Example 4

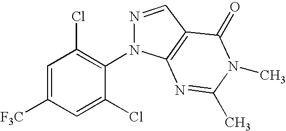 (Process (e))

A mixture of 0.38 g (1.04 mmol) of 1-(2,6-dichloro-4-trifluoromethylphenyl)-6-methyl-1,5-dihydropyrazolo[3,4-d]pyrimidin-4-one, 0.22 g (1.56 mmol) of methyl iodide, 0.22 g (1.56 mmol) of potassium carbonate and 30 ml of dimethylformamide is stirred at 20° C.-25° C. for 6 hours and then concentrated under reduced pressure. The residue is stirred with water and acidified with conc. hydrochloric acid. The resulting crystalline product is isolated by filtration with suction.

This gives 0.30 g (75.5% of theory) of 1-(2,6-dichloro-4-trifluoromethylphenyl)-5,6-dimethyl-1,5-dihydropyrazolo[3,4-d]pyrimidin-4-one of melting point 195° C.

logP (pH 2): 2.69

### Example 5

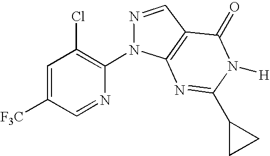 (Process (b))

A mixture of 2.88 g (10 mmol) of 5-amino-1-(3-chloro-5-trifluoromethylpyridin-2-yl)pyrazole-4-carbonitrile, 2.0 g (13 mmol) of cyclopropanecarboxylic anhydride, 4 drops of conc. sulphuric acid and 80 ml of toluene is stirred under reflux for 3 hours and then concentrated under reduced pressure. The residue is triturated with i-propanol and the resulting crystalline product is isolated by filtration with suction.

This gives 1.4 g (38% of theory) of 1-(3-chloro-5-trifluoromethylpyridin-2-yl)-6-cyclopropyl-1,5-dihydropyrazolo[3,4-d]pyrimidin-4-one of melting point 239° C.

logP (pH 2): 2.19

### Example 6

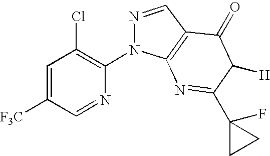 (Process (d))

A mixture of 0.29 g (0.74 mmol) of 5-(1-fluorocyclopropylcarbonylamino)-1-(3-chloro-5-trifluoromethylpyridin-2-yl)pyrazole-4-carboxamide, 20 ml of conc. aqueous ammonia and 30 ml of ethanol is heated under reflux for 6 hours. After cooling to room temperature (about 20° C.), the mixture is extracted with ethyl acetate and the organic phase is dried over sodium sulphate and filtered. From the filtrate, the solvent is carefully distilled off under reduced pressure.

This gives 38 mg (12.5% of theory) of 6-(1-fluorocyclopropyl)-1-(3-chloro-5-trifluoromethylpyridin-2-yl)-1,5-dihydropyrazolo[3,4-d]pyrimidin-4-one of melting point 211° C.

logP (pH 2): 2.29

Analogously to Examples 1 to 6 and in accordance with the general description of the preparation processes according to the invention, it is also possible to prepare, for example, the compounds of the general formulae (I) and (Ia) listed in Table 1 below.

TABLE 1
Examples of compounds of the formulae (I) and (Ia)

| | |
|---|---|
| | (I) |
| 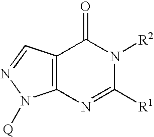 | |
| | (Ia) |
| 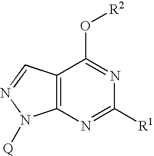 | |

| | | | | Gen. Formula |
|---|---|---|---|---|
| Ex.-No | Q | R¹ | R² | Physical data |
| 7 | 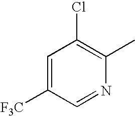 | CH₃ | H | (I) logP = 1.64^{a)} |
| 8 | 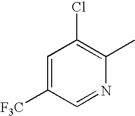 | CH₃ | CH₃ | (I) logP = 1.97^{a)} |
| 9 | 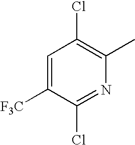 | CH₃ | CH₃ | (I) |
| 10 | 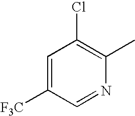 | CH₃ | CH₃ | (Ia) |
| 11 | 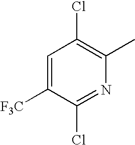 | CH₃ | CH₃ | (Ia) |
| 12 | 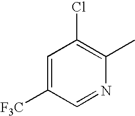 | CHCl₂ | H | (I) logP = 2.39^{a)} |
| 13 | 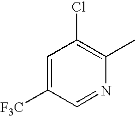 | C₂H₅ | H | (I) logP = 2.39^{a)} |
| 14 | 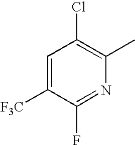 | C₂H₅ | CH₃ | (I) |
| 15 | 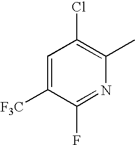 | C₂H₅ | CH₃ | (Ia) |
| 16 | 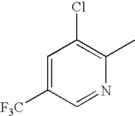 | C₂H₅ | CH₃ | (I) logP = 2.46^{a)} |
| 17 | 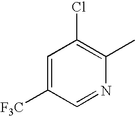 | C₃H₇-i | H | (I) logP = 2.36^{a)} |
| 18 | 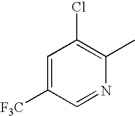 | C₃H₇-i | CH₃ | (I) logP = 2.89^{a)} |
| 19 | 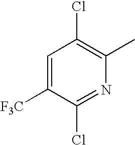 | C₃H₇-i | CH₃ | (I) |
| 20 | 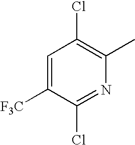 | C₃H₇-i | CH₃ | (Ia) |
| 21 | 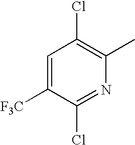 | C₃H₇-i | C₂H₅ | (I) |
| 22 | 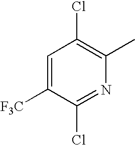 | C₃H₇-i | C₂H₅ | (Ia) |
| 23 | 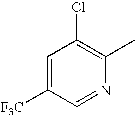 | C₃H₇-n | H | (I) logP = 2.28^{a)} |
| 24 | 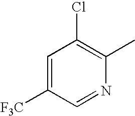 | C₃H₇-n | CH₃ | (I) logP = 2.81^{a)} |
| 25 | 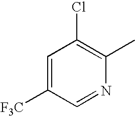 | C₄H₉-i | H | (I) logP = 2.52^{a)} |
| 26 | 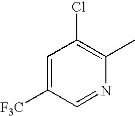 | C₄H₉-t | H | (I) logP = 2.74^{a)} |
| 27 | 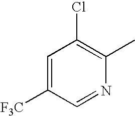 | C₄H₉-t | CH₃ | (Ia) logP = 2.74^{a)} |
| 28 | 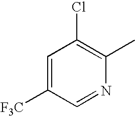 | 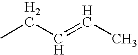 | CH₃ | (I) logP = 2.62^{a)} |
| 29 | 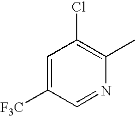 | 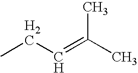 | CH₃ | (I) logP = 2.84^{a)} |
| 30 | 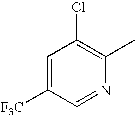 | 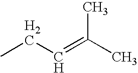 | CH₃ | (Ia) |
| 31 | 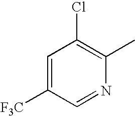 | 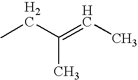 | H | (I) logP = 2.55^{a)} |
| 32 | 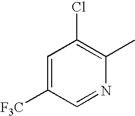 | 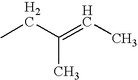 | CH₃ | (I) logP = 2.77^{a)} |
| 33 | 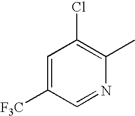 | 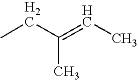 | CH₃ | (Ia) logP = 4.84^{a)} |
| 34 | 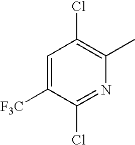 | 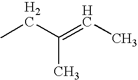 | H | (I) |
| 35 | 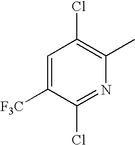 | 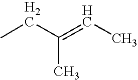 | CH₃ | (I) |
| 36 | 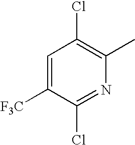 | 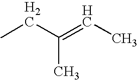 | CH₃ | (Ia) |
| 37 | 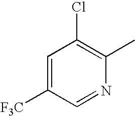 | CF₃ | H | (I) logP = 2.42^{a)} |
| 38 | 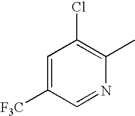 | CF₃ | CH₃ | (I) logP = 3.11^{a)} |
| 39 | 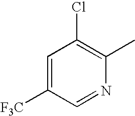 | CF₃ | CH₃ | (Ia) logP = 3.99^{a)} |
| 40 | 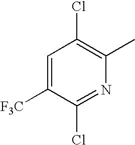 | CF₃ | C₂H₅ | (I) |
| 41 | 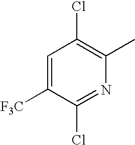 | CF₃ | C₂H₅ | (Ia) |
| 42 | 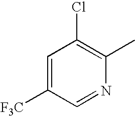 | CF₃ | CH₂COOC₂H₅ | (Ia) logP = 4.05^{a)} |
| 43 | 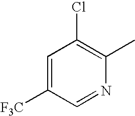 | CF₂Cl | H | (I) logP = 2.53^{a)} |
| 44 | 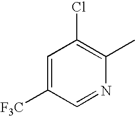 | C₂F₅ | H | (I) logP = 2.84^{a)} |
| 45 | 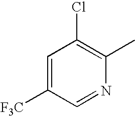 | C₂F₅ | CH₃ | (Ia) |
| 46 | 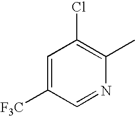 | COOC₂H₅ | CH₃ | (I) logP = 2.74^{a)} |
| 47 | 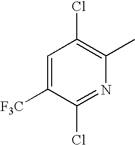 | 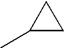 | H | (I) |
| 48 | 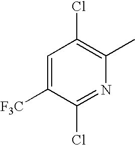 | 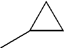 | CH₃ | (I) |
| 49 | 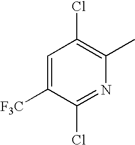 | 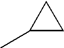 | CH₃ | (Ia) |
| 50 | 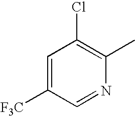 | 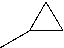 | C₂H₅ | (I) logP = 2.96^{a)} |
| 51 | 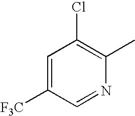 | 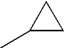 | C₂H₅ | (Ia) logP = 4.42^{a)} |
| 52 | 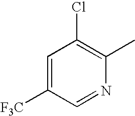 | 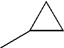 | C₃H₇-i | (Ia) logP = 4.86^{a)} |
| 53 | 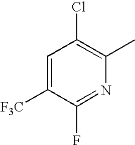 | 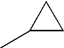 | H | (I) |
| 54 | 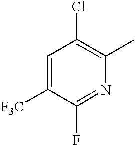 | 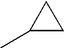 | CH₃ | (I) |
| 55 | 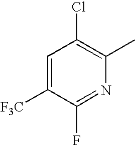 | 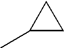 | CH₃ | (Ia) |
| 56 | 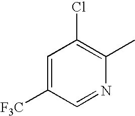 | 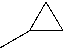 | 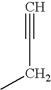 | (I) logP = 2.95^{a)} |
| 57 | 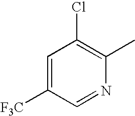 | 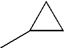 | CHF₂ | (Ia) logP = 4.24^{a)} |
| 58 | 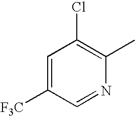 | 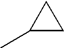 | CH₂COOC₂H₅ | (Ia) logP = 3.88^{a)} |
| 59 | 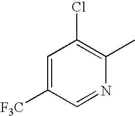 | 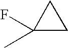 | CH₃ | (I) logP = 2.82^{a)} |
| 60 | 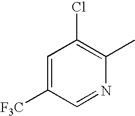 | 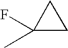 | CH₃ | (Ia) logP = 3.73^{a)} |
| 61 | 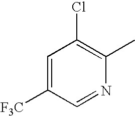 | 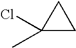 | CH₃ | (I) |
| 62 | 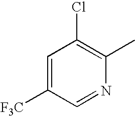 | 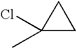 | CH₃ | (Ia) |
| 63 | 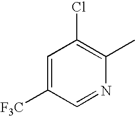 | 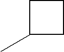 | H | (I) logP = 2.46^{a)} |
| 64 | 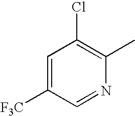 | 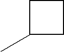 | CH₃ | (I) logP = 3.03^{a)} |
| 65 | 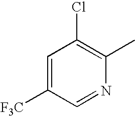 | 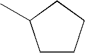 | CH₃ | (I) logP = 3.36^{a)} |
| 66 | 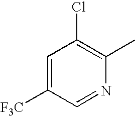 | 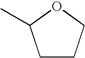 | H | (I) logP = 1.86^{a)} |
| 67 | 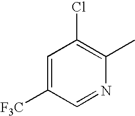 | 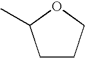 | CH₃ | (I) logP = 2.13^{a)} |
| 68 | 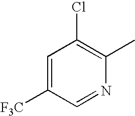 | 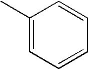 | CH₃ | (I) logP = 2.94^{a)} |
| 69 | 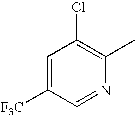 | 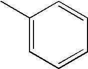 | CH₃ | (Ia) logP = 4.78^{a)} |
| 70 | 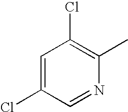 | CH₃ | CH₃ | (I) |
| 71 | 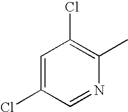 | CH₃ | CH₃ | (Ia) |
| 72 | 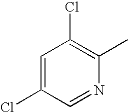 | CF₃ | CH₃ | (Ia) logP = 3.76^{a)} |
| 73 | 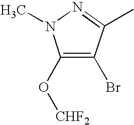 | 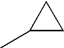 | H | (I) |
| 74 | 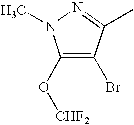 | 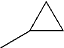 | CH₃ | (I) |
| 75 | 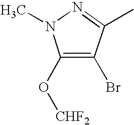 | 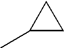 | CH₃ | (Ia) |
| 76 | 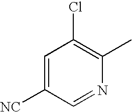 | C₃H₇-i | CH₃ | (I) |
| 77 | 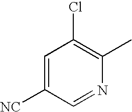 | C₃H₇-i | CH₃ | (Ia) |
| 78 | 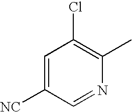 | 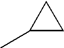 | CH₃ | (I) |
| 79 | 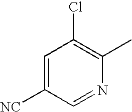 | 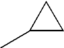 | CH₃ | (Ia) |
| 80 | 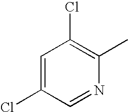 | 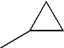 | CH₃ | (I) logP = 2.30^{a)} |
| 81 | 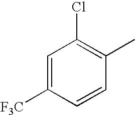 | CH₃ | CH₃ | (I) logP = 2.30^{a)} |
| 82 | 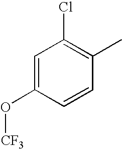 | 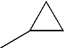 | CH₃ | (I) |
| 83 | 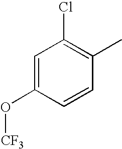 | C₃H₇-i | CH₃ | (Ia) |
| 84 | 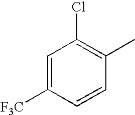 | C₃H₇-i | CH₃ | (I) logP = 3.52^{a)} |
| 85 | 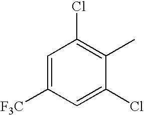 | C₃H₇-i | CH₃ | (I) logP = 3.78^{a)} |
| 86 | 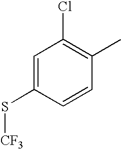 | 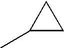 | CH₃ | (I) |
| 87 | 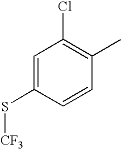 | C₃H₇-i | CH₃ | (I) |
| 88 | 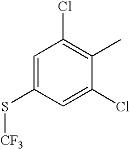 | 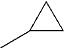 | CH₃ | (I) |
| 89 | 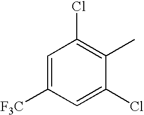 | 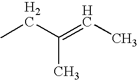 | CH₃ | (I) |
| 90 | 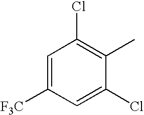 | 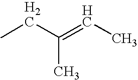 | CH₃ | (Ia) |
| 91 | 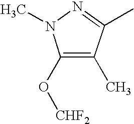 | CH₃ | CH₃ | (I) logP = 1.53^{a)} |
| 92 | 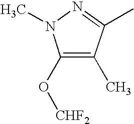 | 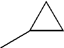 | H | (I) logP = 1.71^{a)} |
| 93 | 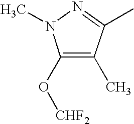 | 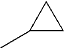 | CH₃ | (I) logP = 2.04^{a)} |
| 94 | 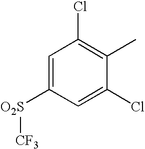 | C₃H₇-i | CH₃ | (I) |
| 95 | 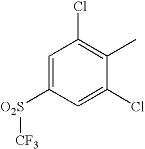 | C₃H₇-i | CH₃ | (Ia) |
| 96 | 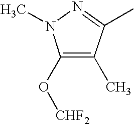 | C₃H₇-i | CH₃ | (I) |
| 97 | 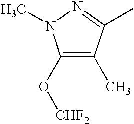 | C₃H₇-i | CH₃ | (Ia) |
| 98 | 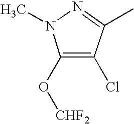 | CH₃ | H | (I) logP = 1.40^{a)}m.p. = 254° C. |
| 99 | 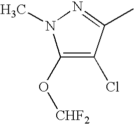 | CH₃ | CH₃ | (I) logP = 1.68^{a)}m.p. = 199° C. |
| 100 | 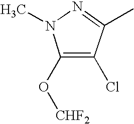 | CH₃ | CH₃ | (Ia) |
| 101 | 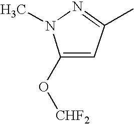 | 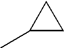 | H | (I) logP = 1.59^{a)} |
| 102 | 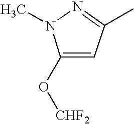 | 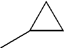 | CH₃ | (I) logP = 1.90^{a)} |
| 103 | 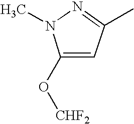 | 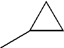 | CH₃ | (Ia) logP = 3.02^{a)} |
| 104 | 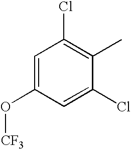 | 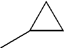 | CH₃ | (I) |
| 105 | 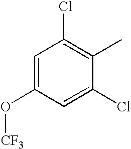 | C₃H₇-i | CH₃ | (Ia) |
| 106 | 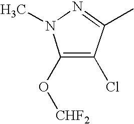 | 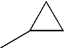 | H | (I) logP = 1.85^{a)}m.p. = 210° C. |
| 107 | 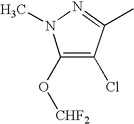 | 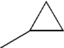 | CH₃ | (I) logP = 2.21^{a)}m.p. = 220° C. |
| 108 | 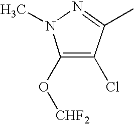 | 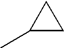 | CH₃ | (Ia) |
| 109 | 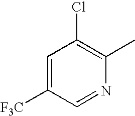 | 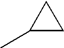 | CH₃ | (I) logP = 2.61^{a)} |
| 110 | 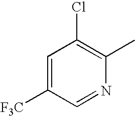 | 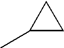 | CH₃ | (Ia) logP = 3.97^{a)} |

The logP values given in the table were determined in accordance with EEC Directive 79/831 Annex V.A8 by HPLC (High Performance Liquid Chromatography) using a reversed-phase column (C 18). Temperature: 43° C.

(a) Mobile phases for the determination in the acidic range: 0.1% phosphoric acid, acetonitrile; linear gradient from 10% acetonitrile to 90% acetonitrile—the corresponding measurement results in Table 1 are marked a).

(b) Mobile phases for the determination in the neutral range: 0.01 molar aqueous phosphate buffer solution, acetonitrile; linear gradient from 10% acetonitrile to 90% acetonitrile—the corresponding measurement results in Table 1 are marked b).

Calibration was carried out using unbranched alkan-2-ones (having 3 to 16 carbon atoms) with known logP values (determination of the logP values by the retention times using linear interpolation between two successive alkanones).

The lambda max values were determined in the maxima of the chromatographic signals using the UV spectra from 200 nm to 400 nm.

The compounds listed above in Table 1 as Examples 109 and 110 can be prepared, for example, as follows:

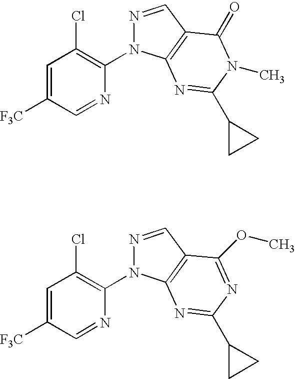

A mixture of 0.80 g (2.25 mmol) of 1-(3-chloro-5-trifluoromethylpyridin-2-yl)-6-cyclopropyl-1,5-dihydropyrazolo[3,4-d]pyrimidin-4-one (cf. Example 5), 0.31 g (2.47 mmol) of dimethyl sulphate, 0.37 g (2.7 mmol) of potassium carbonate and 40 ml of N,N-dimethylformamide is stirred at room temperature (about 20° C.) for 12 hours and then concentrated under reduced pressure. The residue is stirred with water, acidified with conc. hydrochloric acid and extracted with ethyl acetate and the extract is dried over sodium sulphate and filtered. The filtrate is concentrated under reduced pressure. For purification, 0.90 g of the crude product obtained as residue is chromatographed on a silica gel column (toluene/ethyl acetate, 1:1, v/v).

This gives 0.22 g (26% of theory) of 1-(3-chloro-5-trifluoromethylpyridin-2-yl)-4-methoxy-6-cyclopropylpyrazolopyrimidin-4-one of melting point 142° C. (logP (pH 2): 3.97) and 0.56 g (63% of theory) of 1-(3-chloro-5-trifluoromethylpyridin-2-yl)-5-methyl-6-cyclopropyl-1,5-dihydropyrazolo[3,4-d]pyrimidin-4-one of melting point 121° C. (logP (pH 2): 2.61).

Starting Materials of the Formula (II):

### Example (II-1)

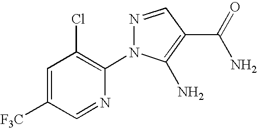

15 g (52 mmol) of 5-amino-1-(3-chloro-5-trifluoromethylpyridin-2-yl)pyrazole-4-carbonitrile are stirred at 60° C. in 150 ml of 98% strength sulphuric acid for 4 hours. The solution is cooled to 20° C. and then stirred with 800 ml of ice-water. The solution is then extracted three times with ethyl acetate and three times with methylene chloride. The combined organic phases are dried over sodium sulphate and filtered. From the filtrate, the solvent is carefully distilled off under reduced pressure.

This gives 14.8 g (94% of theory) of 5-amino-1-(3-chloro-5-trifluoromethylpyridin-2-yl)pyrazole-4-carboxamide of melting point 196° C. (logP (pH 2): 1.46).

Analogously to Example (II-1), it is also possible to prepare, for example, the compounds of the formula (II) listed in Table 2 below.

TABLE 2
Examples of compounds of the formula (II)

| | |
|---|---|
| | (II) |
| 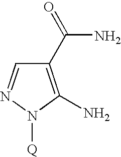 | |

| Ex.-No. | Q | Physical data |
|---|---|---|
| II-2 | 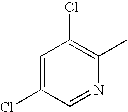 | logP = 1.02^{a)} |
| II-3 | 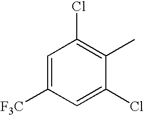 | logP = 1.02^{a)} |
| II-4 | 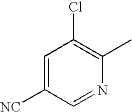 | |
| II-5 | 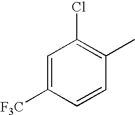 | |

Starting Materials of the Formula (VI):

### Example (VI-1)

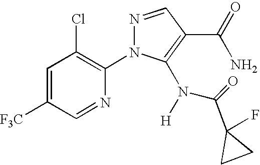

At room temperature (about 20° C.), 0.80 g (31.8 mmol) of sodium hydride (80% in paraffin) is added in portions to 4.86 g (15.9 mmol) of 5-amino-1-(3-chloro-5-trifluoromethyl-pyridin-2-yl)pyrazole-4-carboxamide in 50 ml of acetonitrile, the mixture is stirred for 10 minutes, 3.9 g (31.8 mmol) of 1-fluorocyclopropanecarbonyl chloride are added and the mixture is then stirred at room temperature for 12 hours. The mixture is concentrated under reduced pressure, water is added to the residue and the mixture is then acidified with conc. hydrochloric acid. The resulting crystalline product is isolated by filtration with suction.

This gives 3.1 g (46% of theory) of 5-(1-fluorocyclopropylcarbonylamino)-1-(3-chloro-5-trifluoromethylpyridin-2-yl)pyrazole-4-carboxamide of melting point 204° C. (logP (pH 2): 2.02).

Analogously to Example (VI-1), it is also possible to prepare, for example, the compounds of the formula (VI) listed in Table 3 below.

TABLE 3
Examples of compounds of the formula (VI)

| | |
|---|---|
| | (VI) |
| 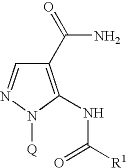 | |

| Ex.-No. | Q | R¹ | Physical data |
|---|---|---|---|
| VI-2 | 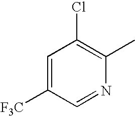 | C₄H₉-t | logP = 2.20^{a)} |
| VI-3 | 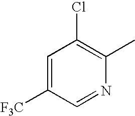 | C(CH₃)₂CH₂Cl | logP = 2.26^{a)} |
| VI-4 | 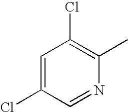 | C₄H₉-t | |
| VI-5 | 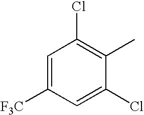 | C₄H₉-t | |
| VI-6 | 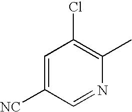 | C₄H₉-t | |
| VI-7 | 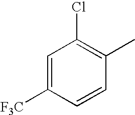 | C₄H₉-t | |

### Use Examples

### Example A

Pre-Emergence Test (Herbicidal Action)

| | |
|---|---|
| Solvent: | 5 parts by weight of acetone |
| Emulsifier: | 1 part by weight of alkylaryl polyglycol ether |

To produce a suitable preparation of active compound, 1 part by weight of active compound is mixed with the stated amount of solvent, the stated amount of emulsifier is added and the concentrate is diluted with water to the desired concentration.

Seeds of the test plants are sown in normal soil. After 24 hours, the soil is sprayed with the preparation of active compound such that the particular amount of active compound desired is applied per unit area. The concentration of active compound in the spray liquor is chosen such that the particular amount of active compound desired is applied in 1000 litres of water per hectare.

After three weeks, the degree of damage to the plants is rated in % damage in comparison to the development of the untreated control. The figures denote:

| | |
|---|---|
| 0% = | no effect (like untreated control) |
| 100% = | total destruction |

In this test, for example, the compounds of Preparation Examples 8, 16, 18, 24, 27, 32, 33, 57, 59, 60, 64, 66, 67, 80, 92 and 109 exhibit strong action against weeds, and some of them are tolerated well by crop plants, such as, for example, maize, wheat and sugar-beet.

### Example B

Post-Emergence Test (Herbicidal Action)

| | |
|---|---|
| Solvent: | 5 parts by weight of acetone |
| Emulsifier: | 1 part by weight of alkylaryl polyglycol ether |

To produce a suitable preparation of active compound, 1 part by weight of active compound is mixed with the stated amount of solvent, the stated amount of emulsifier is added and the concentrate is diluted with water to the desired concentration.

Test plants of a height of 5-15 cm are sprayed with the preparation of active compound such that the particular amounts of active compound desired are applied per unit area. The concentration of the spray liquor is chosen such that the particular amounts of active compound desired are applied in 1000 l of water/ha.

After three weeks, the degree of damage to the plants is rated in % damage in comparison to the development of the untreated control.

The figures denote:

| | |
|---|---|
| 0% = | no effect (like untreated control) |
| 100% = | total destruction |

In this test, for example, the compounds of Preparation Examples 6, 16, 18, 24, 27, 31, 32, 33, 39, 57, 59, 60, 64, 66, 67, 69, 80, 85, 92, 109 and 110 exhibit strong action against weeds, and some of them are tolerated well by crop plants, such as, for example, wheat.

### Example C

Meloidogyne Test (Nematicidal Action)

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

Vessels are filled with sand, solution of active compound, Meloidogyne incognita egg/larvae suspension and lettuce seeds. The lettuce seeds germinate and the plants develop. On the roots, galls are formed.

After the desired period of time, the nematicidal action is determined in % by the formation of galls. 100% means that no galls have been found; 0% means that the number of galls on treated plants corresponds to that of the untreated control.

In this test, for example, the following compounds of the Preparation Examples show good activity: 16 and 80.

1. Compounds of the formula (1) 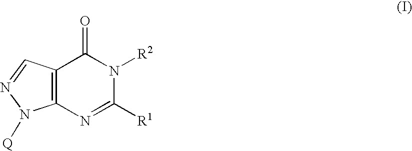
in which
Q represents aryl or heteroaryl, each of which is substituted by at least two identical or different substituents from the group consisting of nitro, cyano, halogen and in each case optionally halogen-substituted C₁-C₆-alkyl, C₁-C₆-alkoxy, C₁-C₆-alkylthio, C₁-C₆-alkylsulphinyl or C₁-C₆-alkylsulphonyl and each of which has up to 10 carbon atoms and, if appropriate, up to 5 nitrogen atoms and/or, if appropriate, one oxygen or sulphur atom,
R¹ represents hydrogen, represents in each case optionally cyano-, halogen- or C₁-C₄-alkoxy-substituted C₁-C₆-alkyl or C₁-C₆-alkoxycarbonyl, or represents in each case optionally halogen-substituted C₂-C₆-alkenyl or C₂-C₆-alkynyl, represents in each case optionally cyano-, halogen- or C₁-C₄-alkyl-substituted C₃-C₆-cycloalkyl or C₃-C₆-cycloalkyl-C₁-C₄-alkyl, represents in each case optionally nitro-, cyano-, halogen-, C₁-C₄-alkyl-, C₁-C₄-haloalkyl-, C₁-C₄-alkoxy- or C₁-C₄-haloalkoxy-substituted aryl or arylalkyl having in each case up to 10 carbon atoms in the aryl group and, if appropriate, up to 4 carbon atoms in the alkyl moiety, or represents optionally nitro-, cyano-, halogen-, C₁-C₄-alkyl-, C₁-C₄-haloalkyl-, C₁-C₄-alkoxy- or C₁-C₄-haloalkoxy-substituted heterocyclyl having up to 10 carbon atoms, up to 5 nitrogen atoms and/or one oxygen or sulphur atom, and
R² represents hydrogen, represents optionally cyano-, halogen-, C₁-C₄-alkoxy- or C₁-C₄-alkoxycarbonyl-substituted C₁-C₆-alkyl or represents in each case optionally halogen-substituted C₂-C₆-alkenyl or C₂-C₆-alkynyl,
except for 1,5-dihydro-6-methyl-1-(2,4,6-trichlorophenyl)-4H-pyrazolo-[3,4-d]-pyrimidin-4-one.

2. Compounds of the formula (Ia) 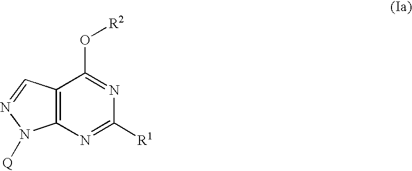
in which
Q, R¹ and R² are as defined in claim 1.

3. Compounds of the formulae (I) and (Ia) according to claim 1 or 2, characterized in that
Q represents aryl having 6 or 10 carbon atoms or heteroaryl having up to 5 carbon atoms, up to 3 nitrogen atoms and/or, if appropriate, one oxygen or sulphur atom, each of which radicals is substituted by at least two identical or different substituents from the group consisting of nitro, cyano, fluorine, chlorine, bromine and C₁-C₄-alkyl, C₁-C₄-alkoxy, C₁-C₄-alkylthio, C₁-C₄-alkylsulphinyl and C₁-C₄-alkylsulphonyl, each of which is optionally substituted by 1 to 3 fluorine and/or chlorine atoms,
R¹ represents hydrogen, represents in each case optionally cyano-, fluorine-, chlorine-, methoxy-, ethoxy-, n- or i-propoxy-substituted C₁-C₅-alkyl or C₁-C₅-alkoxycarbonyl, represents in each case optionally fluorine-, chlorine- and/or bromine-substituted C₂-C₅-alkenyl or C₂-C₅-alkynyl, represents in each case optionally cyano-, fluorine-, chlorine-, methyl- or ethyl-substituted C₃-C₆-cycloalkyl or C₃-C₆-cycloalkyl-C₁-C₃-alkyl, represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted aryl or arylalkyl having in each case 6 or 10 carbon atoms in the aryl group and, if appropriate, up to 3 carbon atoms in the alkyl moiety, or represents optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted heterocyclyl having up to 10 carbon atoms, up to 4 nitrogen atoms and/or one oxygen or sulphur atom, and
R² represents hydrogen, represents optionally cyano-, fluorine-, chlorine-, bromine-, methoxy-, ethoxy-, n- or i-propoxy-, methoxycarbonyl-, ethoxycarbonyl-, n- or i-propoxycarbonyl-substituted C₁-C₅-alkyl or represents in each case optionally fluorine-, chlorine- and/or bromine-substituted C₂-C₅-alkenyl or C₂-C₅-alkynyl.

4. Compounds of the formulae (I) and (la) according to claim 1 or 2, characterized in that,
Q represents phenyl, pyridinyl, pyrimidinyl, pyrazolyl, furyl or thienyl, each of which is substituted by at least two identical or different substituents from the group consisting of nitro, cyano, fluorine, chlorine, bromine, methyl, ethyl, n- or i-propyl, n-, i-, s- or t-butyl, fluoromethyl, chloromethyl, bromomethyl, difluoromethyl, dichloromethyl, trifluoromethyl, trichloromethyl, chlorodifluoromethyl, fluorodichloromethyl, fluoroethyl, chloroethyl, bromoethyl, difluoroethyl, dichloroethyl, chlorofluoroethyl, trifluoroethyl, trichloroethyl, chlorodifluoroethyl, fluorodichloroethyl, tetrafluoroethyl, pentafluoroethyl, methoxy, ethoxy, n- or i-propoxy, difluoromethoxy, trifluoromethoxy, chlorodifluoromethoxy, fluorodichloromethoxy, fluoroethoxy, chloroethoxy, difluoroethoxy, dichloroethoxy, chlorofluoroethoxy, trifluoroethoxy, methylthio, ethylthio, n- or i-propylthio, difluoromethylthio, trifluoromethylthio, chlorodifluoromethylthio, fluorodichloromethylthio, methylsulphinyl, ethylsulphinyl, trifluoromethylsulphinyl, methylsulphonyl, ethylsulphonyl or trifluoromethylsulphonyl,
R¹ represents hydrogen, represents in each case optionally cyano-, fluorine-, chlorine-, methoxy-, ethoxy-, n- or i-propoxy-substituted methyl, ethyl, n- or i-propyl, n-, i-, s- or t-butyl, methoxycarbonyl, ethoxycarbonyl, n- or i-propoxycarbonyl, n-, i-, s- or t-butoxycarbonyl, represents in each case optionally fluorine-, chlorine- and/or bromine-substituted ethenyl, propenyl, butenyl, pentenyl, ethynyl, propynyl, butynyl or pentynyl, represents in each case optionally cyano-, fluorine-, chlorine-, methyl- or ethyl-substituted cyclopropyl, cyclobutyl, cyclopentyl, cyclohexyl, cyclopropylmethyl, cyclobutylmethyl, cyclopentylmethyl or cyclohexylmethyl, represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted phenyl, benzyl or phenylethyl, or represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted pyridinyl, pyrimidinyl, furyl, tetrahydrofuryl or thienyl, and
R² represents hydrogen, represents in each case optionally cyano-, fluorine-, chlorine-, bromine-, methoxy-, ethoxy-, n- or i-propoxy-, methoxycarbonyl-, ethoxycarbonyl-, n- or i-propoxycarbonyl-substituted methyl, ethyl, n- or i-propyl, n-, i-, s- or t-butyl, or represents in each case optionally fluorine-, chlorine- and/or bromine-substituted ethenyl, propenyl, butenyl, pentenyl, ethynyl, propynyl, butynyl or pentynyl.

5. Compounds of the formulae (I) and (Ia) according to claim 1 or 2, characterized in that,
Q represents phenyl which contains at least two identical or different substituents in the 2- and 4-positions and optionally one further substituent in the 6-position, the substituents being selected from the group consisting of nitro, cyano, fluorine, chlorine, bromine, methyl, ethyl, difluoromethyl, dichloromethyl, trifluoromethyl, trichloromethyl, chlorodifluoromethyl, fluorodichloromethyl, methoxy, ethoxy, difluoromethoxy, trifluoromethoxy, chlorodifluoromethoxy, fluorodichloromethoxy, fluoroethoxy, chloroethoxy, difluoroethoxy, dichloroethoxy, chlorofluoroethoxy, trifluoroethoxy, methylthio, ethylthio, difluoromethylthio, trifluoromethylthio, chlorodifluoromethylthio, fluorodichloromethylthio, methylsulphinyl, ethylsulphinyl, trifluoromethylsulphinyl, methylsulphonyl, ethylsulphonyl and trifluoromethylsulphonyl,
R¹ represents hydrogen, represents methyl, ethyl, n- or i-propyl, n-, i- or s-butyl, difluoromethyl, dichloromethyl, trifluoromethyl, trichloromethyl, chlorodifluoromethyl, fluorodichloromethyl, fluoroethyl, chloroethyl, difluoroethyl, dichloroethyl, chlorofluoroethyl, trifluoroethyl, tetrafluoroethyl, pentafluoroethyl, methoxymethyl, ethoxymethyl, methoxyethyl, ethoxyethyl, methoxycarbonyl, ethoxycarbonyl, n- or i-propoxycarbonyl, represents ethenyl, propenyl, butenyl, pentenyl, fluoropropenyl, chloropropenyl, difluoropropenyl, dichloropropenyl, chlorofluoropropenyl, fluorobutenyl, chlorobutenyl, difluorobutenyl, dichlorobutenyl, chlorofluorobutenyl, ethynyl, propynyl, butynyl or pentynyl, represents in each case optionally fluorine-, chlorine- or methyl-substituted cyclopropyl, cyclobutyl, cyclopentyl or cyclohexyl, represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted phenyl, benzyl or phenylethyl, or represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted pyridinyl, pyrimidinyl, furyl, tetrahydrofuryl or thienyl, and
R² represents hydrogen, represents in each case optionally cyano-, fluorine-, chlorine-, methoxy-, ethoxy-, methoxycarbonyl-, ethoxycarbonyl-, n- or i-propoxycarbonyl-substituted methyl, ethyl, n- or i-propyl, or represents in each case optionally fluorine- and/or chlorine-substituted propenyl, butenyl, pentenyl, propynyl, butynyl or pentynyl.

6. Compounds of the formulae (I) and (Ia) according to claim 1 or 2, characterized in that
Q represents pyridin-2-yl which contains at least two identical or different substituents in the 3- and 5-positions and optionally one further substituent in the 6-position, the substituents being selected from the group consisting of nitro, cyano, fluorine, chlorine, bromine, methyl, ethyl, difluoromethyl, dichloromethyl, trifluoromethyl, trichloromethyl, chlorodifluoromethyl, fluorodichloromethyl, methoxy, ethoxy, difluoromethoxy, trifluoromethoxy, chlorodifluoromethoxy, fluorodichloromethoxy, fluoroethoxy, chloroethoxy, difluoroethoxy, dichloroethoxy, chlorofluoroethoxy, trifluoroethoxy, methylthio, ethylthio, difluoromethylthio, trifluoromethylthio, chlorodifluoromethylthio, fluorodichloromethylthio, methylsulphinyl, ethylsulphinyl, trifluoromethylsulphinyl, methylsulphonyl, ethylsulphonyl and trifluoromethylsulphonyl,
R¹ represents hydrogen, represents methyl, ethyl, n- or i-propyl, n-, i- or s-butyl, difluoromethyl, dichloromethyl, trifluoromethyl, trichloromethyl, chlorodifluoromethyl, fluorodichloromethyl, fluoroethyl, chloroethyl, difluoroethyl, dichloroethyl, chlorofluoroethyl, trifluoroethyl, tetrafluoroethyl, pentafluoroethyl, methoxymethyl, ethoxymethyl, methoxyethyl, ethoxyethyl, methoxycarbonyl, ethoxycarbonyl, n- or i-propoxycarbonyl, represents ethenyl, propenyl, butenyl, pentenyl, fluoropropenyl, chloropropenyl, difluoropropenyl, dichloropropenyl, chlorofluoropropenyl, fluorobutenyl, chlorobutenyl, difluorobutenyl, dichlorobutenyl, chlorofluorobutenyl, ethynyl, propynyl, butynyl or pentynyl, represents in each case optionally fluorine-, chlorine- or methyl-substituted cyclopropyl, cyclobutyl, cyclopentyl or cyclohexyl, represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted phenyl, benzyl or phenylethyl, or represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted pyridinyl, pyrimidinyl, furyl, tetrahydrofuryl or thienyl, and
R² represents hydrogen, represents in each case optionally cyano-, fluorine-, chlorine-, methoxy-, ethoxy-, methoxycarbonyl-, ethoxycarbonyl-, n- or i-propoxycarbonyl-substituted methyl, ethyl, n- or i-propyl, or represents in each case optionally fluorine- and/or chlorine-substituted propenyl, butenyl, pentenyl, propynyl, butynyl or pentynyl.

7. Compounds of the formulae (I) and (la) according to claim 1 or 2, characterized in that
Q represents pyrazol-3-yl which contains at least two identical or different substituents in the 1- and 5-positions and optionally one further substituent in the 4-position, the substituents being selected from the group consisting of nitro, cyano, fluorine, chlorine, bromine, methyl, ethyl, difluoromethyl, dichloromethyl, trifluoromethyl, trichloromethyl, chlorodifluoromethyl, fluorodichloromethyl, methoxy, ethoxy, difluoromethoxy, trifluoromethoxy, chlorodifluoromethoxy, fluorodichloromethoxy, fluoroethoxy, chloroethoxy, difluoroethoxy, dichloroethoxy, chlorofluoroethoxy, trifluoroethoxy, methylthio, ethylthio, difluoromethylthio, trifluoromethylthio, chlorodifluoromethylthio, fluorodichloromethylthio, methylsulphinyl, ethylsulphinyl, trifluoromethylsulphinyl, methylsulphonyl, ethylsulphonyl and trifluoromethylsulphonyl,
R¹ represents hydrogen, represents methyl, ethyl, n- or i-propyl, n-, i- or s-butyl, difluoromethyl, dichloromethyl, trifluoromethyl, trichloromethyl, chlorodifluoromethyl, fluorodichloromethyl, fluoroethyl, chloroethyl, difluoroethyl, dichloroethyl, chlorofluoroethyl, trifluoroethyl, tetrafluoroethyl, pentafluoroethyl, methoxymethyl, ethoxymethyl, methoxyethyl, ethoxyethyl, methoxycarbonyl, ethoxycarbonyl, n- or i-propoxycarbonyl, represents ethenyl, propenyl, butenyl, pentenyl, fluoropropenyl, chloropropenyl, difluoropropenyl, dichloropropenyl, chlorofluoropropenyl, fluorobutenyl, chlorobutenyl, difluorobutenyl, dichlorobutenyl, chlorofluorobutenyl, ethynyl, propynyl, butynyl or pentynyl, represents in each case optionally fluorine-, chlorine- or methyl-substituted cyclopropyl, cyclobutyl, cyclopentyl or cyclohexyl, represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, n-, i-, s- or t-butoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted phenyl, benzyl or phenylethyl, or represents in each case optionally nitro-, cyano-, fluorine-, chlorine-, bromine-, methyl-, ethyl-, n- or i-propyl-, n-, i-, s- or t-butyl-, difluoromethyl-, dichloromethyl-, trifluoromethyl-, chlorodifluoromethyl-, fluorodichloromethyl-, methoxy-, ethoxy-, n- or i-propoxy-, difluoromethoxy-, trifluoromethoxy-, chlorodifluoromethoxy-, fluoroethoxy-, chloroethoxy-, difluoroethoxy-, dichloroethoxy-, chlorofluoroethoxy-, chlorodifluoroethoxy- or trifluoroethoxy-substituted pyridinyl, pyrimidinyl, furyl, tetrahydrofuryl or thienyl, and
R² represents hydrogen, represents in each case optionally cyano-, fluorine-, chlorine-, methoxy-, ethoxy-, methoxycarbonyl-, ethoxycarbonyl-, n- or i-propoxycarbonyl-substituted methyl, ethyl, n- or i-propyl, or represents in each case optionally fluorine- and/or chlorine-substituted propenyl, butenyl, pentenyl, propynyl, butynyl or pentynyl.

8. Process for preparing compounds of the formulae (I) and (Ia) according to claim 1 or 2, characterized in that
a) compounds of the formula (II) 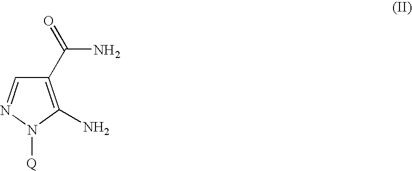
in which
Q is as defined in claim 1
are reacted with compounds of the formula (III)
R¹—(OR′)₃ (III)
in which
R¹ is as defined in claim 1 and
R′ represents alkyl,
if appropriate in the presence of one or more reaction auxiliaries and if appropriate in the presence of one or more diluents,
or that
(b) compounds of the formula (IV) 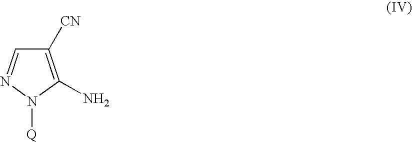
in which
Q is as defined in claim 1
are reacted with compounds of the formula (V) 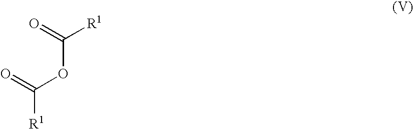
in which
R¹ is as defined in claim 1,
if appropriate in the presence of one or more reaction auxiliaries and if appropriate in the presence of one or more diluents,
or that
(c) compounds of the formula (II) 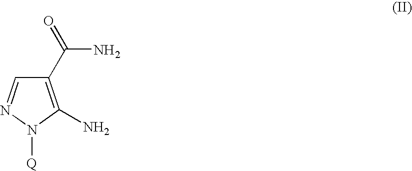
in which
Q is as defined in claim 1,
are reacted with compounds of the formula (V) 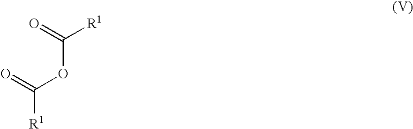
in which
R¹ is as defined in claim 1,
if appropriate in the presence of one or more reaction auxiliaries and if appropriate in the presence of one or more diluents,
or that
(d) compounds of the formula (VI) 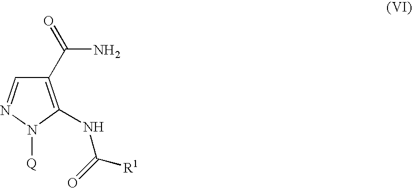
in which
Q and R¹ are as defined in claim 1,
are reacted, if appropriate in the presence of one or more condensing agents and if appropriate in the presence of one or more diluents,
or that
(e) compounds of the formula (Ib) 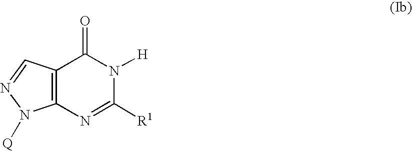
in which
Q and R¹ are as defined in claim 1,
are reacted with alkylating, alkenylating or alkynylating agents of the formula (VII)
X—R² (VII)
or of the formula (VII)
R²—O—SO₂—O—R² (VIII)
where in each case
R² is as defined in claim 1 and
X represents halogen,
if appropriate in the presence of one or more reaction auxiliaries and if appropriate in the presence of one or more diluents.

9. Compounds of the formula (II) 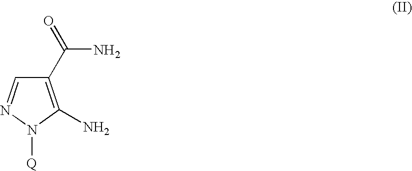
in which
Q is as defined in claim 1.

10. Compounds of the formula (VI) 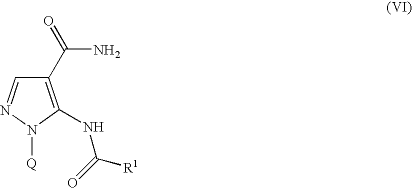
in which
Q and R¹ are as defined in claim 1.

11. Compounds of the formula (Ib) 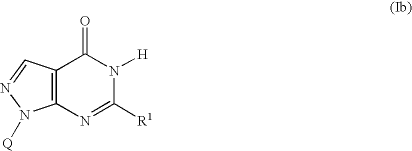
in which
Q and R¹ are as defined in claim 1.

12. Crop treatment agents, characterized in that they comprise at least one compound according to any of claims 1 to 7 and customary extenders and/or surfactants.

13. Use of at least one compound according to any of claims 1 to 7 or of a composition according to claim 12 for controlling unwanted plants and/or nematodes.

14. Method for controlling unwanted plants and/or nematodes, characterized in that at least one compound according to any of claims 1 to 7 or a composition according to claim 12 is allowed to act on the plants and/or nematodes and/or their habitat.
